SEQUENCE LISTING (41 sequences)

SEQ ID NO: 1 - DNA, 300 bp, synthetic construct
  source 1..300: mol_type = other DNA
ttgttacctctgactcccagttgtttaataaaccatattggttacataaggcacagggtcataacaatggtgtttgctggcataatcaattatttgttactgtggtagataccactcccagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagta

SEQ ID NO: 2-7 - skipped (6 entries)

SEQ ID NO: 8 - RNA, 20 nt, synthetic construct
  source 1..20: mol_type = other RNA
acaatatgtgcttctacaca

SEQ ID NO: 9-20 - skipped (12 entries)

SEQ ID NO: 21 - RNA, 598 nt, synthetic construct
  source 1..598: mol_type = other RNA
tgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacc

SEQ ID NO: 22 - RNA, 20 nt, synthetic construct
  source 1..20: mol_type = other RNA
cgaagaacgctgaagcgctg

SEQ ID NO: 23 - RNA, 22 nt, synthetic construct
  source 1..22: mol_type = other RNA
tagcttatcagactgatgttga

SEQ ID NO: 24 - RNA, 20 nt, synthetic construct
  source 1..20: mol_type = other RNA
tgaatcaacatcagtctgat

SEQ ID NO: 25 - DNA, 46 bp, synthetic construct
  source 1..46: mol_type = other DNA
gaagttcatgtttctgaatcaacatcagtctgataagctattcaag

SEQ ID NO: 26 - DNA, 18 bp, synthetic construct
  source 1..18: mol_type = other DNA
ttcagaaacatgaacttc

SEQ ID NO: 27 - DNA, 34 bp, synthetic construct
  source 1..34: mol_type = other DNA
aattatttgttactgtggtagataccactcccag

SEQ ID NO: 28 - DNA, 35 bp, synthetic construct
  source 1..35: mol_type = other DNA
cacaactgaaaaataaactgcaaatcatattcctc

SEQ ID NO: 29 - DNA, 18 bp, synthetic construct
  source 1..18: mol_type = other DNA
agatcacattggcacccg

SEQ ID NO: 30 - DNA, 20 bp, synthetic construct
  source 1..20: mol_type = other DNA
ccattgccagccattctagc

SEQ ID NO: 31 - DNA, 41 bp, synthetic construct
  source 1..41: mol_type = other DNA
tgctcccttctgcgtagaagccaatgctgcaatcgtgctac

SEQ ID NO: 32 - DNA, 39 bp, synthetic construct
  source 1..39: mol_type = other DNA
ggcggcagtcaagcctcttccctactgctgcctggagtt

SEQ ID NO: 33 - DNA, 24 bp, synthetic construct
  source 1..24: mol_type = other DNA
gcaatgttgttccttgaggaagtt

SEQ ID NO: 34 - DNA, 24 bp, synthetic construct
  source 1..24: mol_type = other DNA
gttcctcatcacgtagtcgcaaca

SEQ ID NO: 35 - RNA, 20 nt, synthetic construct
  source 1..20: mol_type = other RNA
tgaacctcatcacgtagtcg

SEQ ID NO: 36 - DNA, 46 bp, synthetic construct
  source 1..46: mol_type = other DNA
gaagttcatgtttctgaagtagatatggcagcactaatctaatatg

SEQ ID NO: 37 - DNA, 18 bp, synthetic construct
  source 1..18: mol_type = other DNA
ttcagaaacatgaacttc

SEQ ID NO: 38 - skipped

SEQ ID NO: 39 - RNA, 20 nt, synthetic construct
  source 1..20: mol_type = other RNA
tgaagtagatatggcagcac

SEQ ID NO: 40 - RNA, 20 nt, synthetic construct
  source 1..20: mol_type = other RNA
tgaagtagatatggcagcac

SEQ ID NO: 41 - DNA, 88 bp, synthetic construct
  source 1..88: mol_type = other DNA
gcgctaatacgactcactataggggcgtcattgatgcatattagattagtgctgccatatctacaggtcgactttcaagaattcatat

(19) United States
(12) Patent Application Publication
(10) Pub. No.: US 2025/0333778 A1
(43) Pub. Date: Oct. 30, 2025

(54) METHOD FOR DETECTING TARGET NUCLEIC ACID BY CLEAVING NON-NATURAL SEQUENCE USING CAS12 PROTEIN

(71) Applicants: Orange Biotech Limited, HONG KONG (HK); THE GOVERNING COUNCIL OF THE UNIVERSITY OF TORONTO, Toronto (CA)

(72) Inventors: Xin LIU, HONG KONG (HK); Idorenyin A. IWE, Toronto (CA); Shuhuai YAO, HONG KONG (HK); Keith PARDEE, Toronto (CA); Ariel CORSANO, Toronto (CA)

(21) Appl. No.: 19/068,591

(22) Filed: Mar. 3, 2025

Related U.S. Application Data
(60) Provisional application No. 63/633,180, filed on Apr. 12, 2024.

(51) Int. Cl. C12Q 1/6823; C12N 15/10; C12Q 1/34; C12Q 1/6806; C12Q 1/686; C12Q 1/6876

(52) U.S. Cl. CPC C12Q 1/6823; C12N 15/1096; C12Q 1/34; C12Q 1/6806; C12Q 1/686; C12Q 1/6876; G01N 2333/922

(57) ABSTRACT

A method for detecting a target nucleic acid includes cleaving a non-natural sequence using a Cas12 protein. The Cas12 protein belongs to a Cas12a/Cas12b protein family. The non-natural sequence includes a chimeric sequence with a ribonucleotide and a deoxyribonucleotide, which can be made into a probe for nucleic acid detection, and its detection effect is better than a conventional ssRNA probe, and may be equivalent to or even better than an ssDNA probe. In addition, a system for detecting a target nucleic acid includes a chimeric sequence, a Cas12 protein and crRNA. Pre-amplification can increase the detection limit of the system to a single-molecule level. The Cas12-chimeric sequence detection system can be used not only on a microfluidic chip, but also in clinical testing. The coordinated use of the Cas12 protein and chimeric sequence optimizes the CRISPR/Cas12 detection system and expands use of Cas12a protein and non-natural sequence.

### CROSS-REFERENCE TO RELATED APPLICATIONS

The present application claims priority to US prior provisional application No. 63/633,180 filed on Apr. 12, 2024, the claims, specification, drawings of specification, and abstract of which are incorporated herein by reference in their entirety as part of the present invention.

### REFERENCE TO SEQUENCE LISTING SUBMITTED ELECTRONICALLY

The nucleotide and/or amino acid sequences disclosed in this application are presented in a separate XML file, “20250331 Cas12-US application.xml”, created on Mar. 31, 2025, which has a size of 60 kilobytes. The content of “20250331 Cas12-US application.xml” is incorporated herein by reference in its entirety.

### BACKGROUND OF THE INVENTION

### Field of the Invention

The present invention belongs to the technical field of biology, and in particular relates to a method and kit for detecting a target nucleic acid by cleaving a non-natural sequence using a Cas12 protein.

### Description of the Related Art

CRISPR (Clustered Regularly Interspaced Short Palindromic Repeats) gene editing technology is widely used in various fields due to its outstanding editability and simple operation steps. The research on a Cas protein in a CRISPR/Cas editing system has brought breakthrough progress in the field of nucleic acid diagnosis and is considered to be the next generation of nucleic acid detection tools. The trans-cleavage activity of a CRISPR Class 2 Cas protein is the basis of protein diagnostic technology. The CRISPR Class 2 Cas protein is composed of two families, Cas12 and Cas13. The trans-cleavage activity refers to the non-specific cleavage of nucleic acid sequences.

The principle of this technology is as follows: under the guidance of CRISPR RNA (crRNA), the Cas protein can specifically identify, bind and cleave a target nucleic acid; and once the Cas protein identifies a target sequence thereof, its trans-cleavage activity is immediately activated, that is, it can non-specifically cleave other adjacent nucleic acid sequences. Based on this characteristic of the Cas protein, the adjacent nucleic acid sequence is usually designed as an oligonucleotide probe with a fluorescent group at one end and a fluorescent quenching group at the other end to reflect whether the Cas protein detects the presence of the target nucleic acid under the guidance of the crRNA. Furthermore, due to the high programmability of the crRNA, it can be used for guiding the Cas protein to target any sequence of interest. Under normal circumstances, the fluorescence emitted by the fluorescent group is quenched by the quenching group since the fluorescent group is close to the quenching group. However, when the Cas protein identifies the target nucleic acid under the guidance of the crRNA, it trans-cleaves the probe, so that the fluorescent group on the probe is separated from the quenching group, and the fluorescent group normally emits fluorescence, which is detected by an instrument, thereby achieving the purpose of indirect detection of the target nucleic acid. In addition, any physical or chemical visualization method is suitable for detecting probe fragmentation caused by the trans-cleavage activity of the Cas protein.

However, not all members of the Cas protein family have trans-cleavage activity. Among these members, Cas12 and Cas13 proteins in Class 2 of the Cas protein family have become hot spots in nucleic acid detection because they have both cis-cleavage activity and trans-cleavage activity under the guidance of the CRISPR RNA (crRNA). Interestingly, the cleavage manners of the Cas12 protein and the Cas13 protein are not exactly the same. Under the guidance of guided-RNA (gRNA) or the crRNA, the Cas12 protein (e.g. Cas12a and Cas12b proteins) also cleaves non-specific single-stranded DNA (ssDNA) while cleaving double-stranded DNA (dsDNA) having a specific sequence. A CRISPR/Cas12 gene editing system can detect the target nucleic acid with high specificity and sensitivity, leading to the development of systems such as HOLMES (One-HOur Low-cost Multipurpose highly Efficient System) and DETECTR (DNA Endonuclease-Targeted CRISPR Trans Reporter) . . . . In contrast, the Cas13 needs the assistance of the gRNA to perform a nucleic acid cleavage function, but it can non-specifically cleave the single-stranded RNA (ssRNA). The CRISPR/Cas13 system allows highly accurate and efficient RNA molecule detection, exemplified by the SHERLOCK platform (Specific High-sensitivity Enzymatic Reporter UnLOCKing).

Currently, the CRISPR/Cas12 system is hailed as a rising star among CRISPR/Cas technologies due to its similarity to the CRISPR/Cas9 system in targeting DNA sequences, with the added ability to activate trans-cleavage activity Therefore, many studies have revealed the exciting new functions of the Cas12 protein, which provides new possibilities for the development of subsequent diagnostic fields. Current studies have shown that the CRISPR/Cas12a system and the CRISPR/Cas12b system can identify a DNA sequence and trans-cleave ssDNA sequences modified with fluorescent labels, magnetism, colors and other modifications; the Cas12a2 protein and the Cas12g protein can trans-cleave the ssRNA, the ssDNA and the dsDNA; moreover, some Cas12 variants can directly target RNA with the assistance of a DNA sequence; and a LbCas12a protein is found to have RNase activity, although its RNA trans-cleavage activity is much lower than its DNA activity. However, it is not clear whether the weak RNase activity of the Cas12 protein can be applied to the field of nucleic acid detection to have similar performance as the DNase activity

To address this issue, identifying the preferred ssRNA sequences to optimize RNase activity or exploring unknown nuclease activities can help expand the system's specific diagnostic and detection applications.

### BRIEF SUMMARY OF THE INVENTION

Aiming at the aforementioned traditional problems, the present invention provides a method or kit and system for detecting the presence or quantity of a target nucleic acid by cleaving a non-natural sequence using a Cas12 protein. This invention belongs to the field of biological technology.

In one aspect, the present invention involves use of a Cas12 protein in preparation of an agent for trans-cleaving a non-natural sequence to detect a target nucleic acid, where the non-natural sequence includes any one or more of the following:
(1) a sequence containing both a deoxynucleotide and a ribonucleotide;
(2) a sequence containing a natural or non-natural deoxynucleotide and/or ribonucleotide, where the deoxynucleotide and/or ribonucleotide bears an artificially created modification that does not exist under natural conditions; and
(3) a sequence containing a deoxynucleotide and/or ribonucleotide, where a backbone composed of the deoxynucleotide and/or ribonucleotide bears an artificially created modification that does not exist under natural conditions.

In a second aspect of the present invention, the present invention provides a kit for detecting a target nucleic acid, including: a Cas12 protein for binding to the target nucleic acid, and a non-natural or non-naturally occurring nucleic acid sequence. The non-natural sequence includes one or more of the following sequences:
(1) a sequence containing both a deoxynucleotide and a ribonucleotide;
(2) a sequence containing a natural or non-natural deoxynucleotide and/or ribonucleotide, where the deoxynucleotide and/or ribonucleotide bears an artificially created modification that does not exist under natural conditions; and
(3) a sequence containing a deoxynucleotide and/or ribonucleotide, where a backbone composed of the deoxynucleotide and/or ribonucleotide bears an artificially created modification that does not exist under natural conditions.

In some embodiments, the kit further includes an agent necessary for amplifying the target nucleic acid, including an enzyme, an inorganic salt, etc necessary for amplification. In some embodiments, a target nucleic acid amplification manner includes thermal cycling-based PCR or isothermal amplification, and the isothermal amplification includes loop-mediated isothermal amplification (LAMP), recombinase polymerase amplification (RPA), recombinase-aided amplification (RAA), and similar methods. All agents or components that can amplify the target nucleic acid can be used as an embodiment of the present invention, such as a primer, a probe sequence that binds to a target nucleic acid, etc.

Here, the target nucleic acid is a nucleic acid of interest for detection or diagnosis. The target nucleic acid is generally a natural nucleic acid sequence or a partial sequence produced by synthesis, nucleic acid amplification, and the like methods, e.g. tissues of human body, microorganisms such as viruses, bacteria, fungi, and also human or mammalian cells, etc.

In some embodiments, the target nucleic acid is DNA or RNA.

In some embodiments, among the target nucleic acids, the DNA or RNA includes double strands or a single strand.

In some embodiments, among the target nucleic acids, the DNA is double-stranded and the RNA is single-stranded.

In some embodiments, the artificially synthesized target nucleic acid includes a target nucleic acid, a DNA helper in an RAPID system.

In a third aspect of the present invention, the present invention provides a method for detecting the presence or quantity of a target nucleic acid, including: allowing a Cas12 protein to bind with a target nucleic acid to bind, and meanwhile allowing the Cas12 protein or enzyme to trans-cleave a non-natural sequence, so as to detect or identify the presence or quantity of the target nucleic acid from the number of the cleaved non-natural sequence.

In some embodiments, the non-natural sequence includes one or more of the following sequences:
(1) a sequence containing both a deoxynucleotide and a ribonucleotide;
(2) a sequence containing a natural or non-natural deoxynucleotide and/or ribonucleotide, where the deoxynucleotide and/or ribonucleotide bears an artificially created modification that does not exist under natural conditions; and
(3) a sequence containing a deoxynucleotide and/or ribonucleotide, where a backbone composed of the deoxynucleotide and/or ribonucleotide bears an artificially created modification that does not exist under natural conditions.

In some embodiments, the non-natural sequence includes a label, and the presence or quantity of the target nucleic acid is detected by detecting the amount or quantity of the label. The label includes fluorescence or any other labeling substances.

In some embodiments, the Cas12 protein belongs to a Cas12a/Cas12b protein family.

In some embodiments, the non-natural nucleic acid sequence includes a chimeric sequence composed of a ribonucleotide and a deoxyribonucleotide. Such a sequence can be made into a probe for nucleic acid detection. The detection effect of the chimeric sequence is better than that of a conventional ssRNA probe, and in certain cases is comparable to or even better than that of an ssDNA probe. The non-natural nucleic acid also includes a xeno nucleic acid (XNA), a chimeric sequence, and a hybridized sequence.

In some embodiments, a natural target nucleic acid may or may not be amplified. In some embodiments, the target nucleic acid is pre-amplified, and then tested by the method or system of the present invention. The present invention also demonstrates that the pre-amplification of the target nucleic acid can increase the limit of detection of the system to a single-molecule level. The present invention further verifies that the Cas12-chimeric sequence detection system can be used not only on a microfluidic chip, but also in clinical testing. In summary, the combined use of the Cas12 protein and chimeric sequence optimizes the CRISPR/Cas12 detection system and expands the use of the Cas12 protein and the non-natural sequence.

In some embodiments, the non-natural or non-naturally occurring nucleic acid of the present invention includes a non-natural or non-naturally occurring nucleic acid sequence in a broad sense, and also includes a non-natural or non-naturally occurring nucleic acid sequence in a narrow sense. In some embodiments, the non-natural or non-naturally occurring nucleic acid of the present invention is the non-natural nucleic acid sequence in the broad sense. In some embodiments, the meanings of the “non-natural sequence” or the “non-naturally occurring sequence” are interchangeable, and they refer to nucleic acid sequences incapable of being produced or incapable of being stably inherited during a long evolutionary process in nature, i.e., other sequences other than natural or naturally occurring DNA and RNA. The natural or naturally occurring nucleic acid sequence (e.g. RNA or DNA) refers to a nucleic acid sequence that can be produced or stably inherited during a long evolutionary process in nature.

In some embodiments, the non-natural sequence refers to a sequence that is different from a natural or naturally occurring DNA/RNA and is created by artificially modifying the components or internal structure of the natural DNA/RNA on the basis of an existing natural DNA/RNA sequence, also known as a xeno nucleic acid (XNA). In some embodiments, the artificial modification of the components of the DNA/RNA includes, but is not limited to, changing the combination manner of the components (i.e., the deoxynucleotides and the nucleotides can appear simultaneously in the same sequence), and making non-natural and artificially created modifications to the internal components of the nucleotides such as pentoses, bases or phosphate groups, etc. The artificial modification methods include, but are not limited to: changing the type of a glycosyl group, introducing an organic polymer and/or a halogen, and a combination of several or multiple of a plurality of methyl or/acetyl modifications. In some embodiments, the artificial base modification can regulate the strength and specificity of base pairing, and the modification of the glycosyl group also has a significant effect on the properties of nucleic acids, e.g. double-strand formation ability, nuclease resistance, and toxicity to cells and animals. In some embodiments, the artificial modification of the internal structure of the DNA/RNA includes, but is not limited to change in a nucleic acid backbone structure (e.g. a thiophosphate), introduction of a new artificial nucleoside (e.g. deoxyuridine), modification of pentoses (e.g. the pentoses of glycol nucleic acids), and deoxy and non-deoxy modifications, etc. In some embodiments, the artificial modification of the phosphodiester backbone can improve the nuclease resistance and pharmacokinetic properties. In a narrow sense, the non-natural sequence refers to a sequence composed of a deoxynucleotide and a ribonucleotide, or a nucleic acid sequence containing a modified deoxynucleotide and/or ribonucleotide and/or sugar-phosphate backbone that is artificially created under non-natural conditions. The meaning of the “non-natural sequence” or “non-naturally occurring sequence” described in the present invention includes both the “non-natural sequence” or “non-naturally occurring sequence” in a broad sense and the “non-natural sequence” or “non-naturally occurring sequence” in a narrow sense.

In some embodiments, the xeno nucleic acid (XNA) can store gene information, replicate, and even evolve, just like natural DNA and RNA, but is artificially created or produced, rather than produced by natural evolution. The term “synthetic” in the present invention means artificial synthesis, rather than production in an evolutionary process in nature. The so-called “artificial synthesis” includes synthesis participated and managed by humans, for example synthesis of the “non-natural sequence” or “non-naturally occurring sequence” as defined in the present invention by humans through a machine or in an artificial intelligence (AI) manner.

In some embodiments, the non-natural nucleic acid sequence includes a (rUA)ₙ nucleic acid sequence, where n is any natural integer, and for example, n can be any number from 10,000 to 100 million, e.g., a natural length of 1, 2, 3, 4, 5, 6, 7, 8, 9, 10, 11, 12, 13, 14, 15, etc. In some embodiments, non-natural sequences (e.g., chimeric sequences) in the following forms, such as rUArUArUA, where “rU” represents a uracil ribonucleotide (which only occurs in RNA in vivo) and “A” represents an adenine deoxynucleotide (which only occurs in DNA in vivo), demonstrate how synthetic biology combines natural components to create a novel genetic system. In an embodiment, the non-natural sequence includes an RNA or DNA sequence with (rUA)ₙ units. For example, the natural sequence includes (rUA)ₙ, and for example, a DNA sequence includes a (rUA) n unit, where n is any natural integer. In some embodiments, the natural DNA or RNA sequence includes a nucleic acid sequence with a (rUA)ₙ unit, thereby becoming a non-natural sequence or a chimeric sequence.

A basic unit of traditional natural DNA is a deoxyribonucleotide. The deoxyribonucleotide is composed of a base, a deoxyribose and a phosphate. The basic building block of DNA is a deoxyribonucleotide, which is of four types, depending on the base it contains: A (adenine), T (thymine), G (guanine), and C (cytosine). In contrast, the basic building block of natural RNA is a ribonucleotide, which is composed of one molecule of phosphoric acid, one molecule of ribose, and one molecule of nitrogenous base. There are four types of nitrogenous bases: adenine (A), uracil (U), guanine (G) and cytosine (C), respectively.

These chimeric sequences are intermediate states between natural nucleic acids and engineered alternatives, providing new possibilities for the research and application of the present invention. In synthetic biology, non-natural nucleic acids can be used in editable cells, providing new ideas for more stable gene therapies, biosensors, and synthetic organisms. They also have certain potential in the field of biotechnology, where they can be used as probes with a longer shelf life and better durability in molecular diagnostics or as new components of gene editing systems such as CRISPR. Moreover, XNAs can also be used for constructing nanoscale scaffolds or as components of programmable biomolecular systems. More importantly, non-natural nucleic acids can achieve the goal of constructing synthetic cells with completely new genetic information, fundamentally expanding human understanding of lives and potential forms thereof.

In some embodiments, the Cas12 belongs to Class 2 V-type RNA-guided endonucleases, which include Cas12a and Cas12b, etc. The most studied nuclease in this protein family is the nuclease Cas12a. Currently, the most commonly used Cas12a protein is derived from a BV3L6 strain of the genus Acidaminococcus and the bacteria of the family Lachnospiraceae (LbCas12a). The Cas12a protein has been widely applied in multiple species including bacterial, yeast, plant, and human cells.

The Cas12 has both cis-cleavage and trans-cleavage activities. When the Cas12 specifically identifies and cleaves the target nucleic acid (this process is cis-cleavage), such a process can activate its trans-cleavage activity and can cleave any other single-stranded natural DNA in the system into fragments (this process is trans-cleavage) in a short period of time. This characteristic of Cas12 is utilized to detect various target DNA in the prior art. However, in the present invention it has been found that this system can be utilized to trans-cleave non-natural or non-naturally occurring DNA sequences.

The Cas12 not only can cleave single-stranded DNA, but also can cleave a single-stranded RNA through an RNase (cis or trans), but its RNA cleaving activity is relatively weaker, so it is believed in the prior art that it is difficult to construct a target nucleic acid detection system by trans-cleaving natural RNA with the Cas12. However, it has been proved through a large number of studies in the present invention that the Cas12 can achieve accurate detection of the target nucleic acid by trans-cleaving non-natural sequences through an RNase, thereby greatly broadening the application of the Cas12 in the field of target nucleic acid detection.

The “trans-cleavage” described in the present invention refers to the property that after being activated by the target nucleic acid, the Cas12 protein can non-specifically cleave any other adjacent nucleic acid sequence, including DNA, RNA or non-natural sequences. The target nucleic acid includes natural DNA or RNA in a sample.

In some specific embodiments, the non-natural sequence contains both a deoxynucleotide and a ribonucleotide.

It can be understood that the natural DNA or RNA only contains the deoxynucleotide or the ribonucleotide, so that the non-natural sequence is neither a DNA sequence nor an RNA sequence.

In some specific embodiments, the deoxynucleotides and ribonucleotides in the non-natural sequence are arranged alternately at intervals. That is, a deoxynucleotide is followed by a ribonucleotide, or a ribonucleotide is followed by a deoxynucleotide. It can be understood that the non-natural sequence does not contain two deoxynucleotides or two ribonucleotides connected by a natural backbone, e.g.: DNA: TT, RNA: UU and the like situations.

Further, the Cas12 protein utilizes its RNase activity to trans-cleave the non-natural sequence.

Studies have shown that the Cas12 protein has stronger trans-cleavage DNase activity and weaker RNase activity. Therefore, there has been no report on the application of this RNase cleavage ability to research or progress in nucleic acid detection. The present invention investigates the RNAse activity of LbCas12a. Compared with a traditional system, the overall performance of the LbCas12a-ssRNA system still has much room for improvement. In some methods, in order to improve the efficiency of the Cas12 protein in trans-cleavage of natural RNA, we not only perform non-natural modifications on a natural RNA sequence, but also create a non-natural sequence that contains both a deoxynucleotide and a ribonucleotide; and before this, no one has ever linked the RNase activity of the Cas12 protein, the non-natural sequence, and target nucleic acid detection together.

The present invention designs 1 RNA sequence with a modified thiophosphate bond (e.g.: T*A*rArU*G*C) and 2 non-natural chimeric sequences (rUArUArUA and ArUArUArU), and uses a conventional LbCas12a protein to trans-cleave them. It is found that the aforementioned 3 non-natural sequences can be trans-cleaved by LbCas12a, and the detection effects on them are better than that of ssRNA (rUrUrUrUrUrU or rArArArArArA), and the efficiency of the Cas12 in cleaving 2 non-natural sequences is comparable to that of a positive control ssDNA.

In summary, the present invention proves for the first time that the Cas12a can utilize its RNase activity to trans-cleave non-natural sequences, and this feature can be applied to nucleic acid detection; and it also proves that the modification of natural RNA can improve the trans-cleavage efficiency of the Cas12a protein, and in turn achieve a very high signal-to-noise ratio during the detection process, but predecessors have not utilized this feature to apply for related patents.

In another aspect, the present invention provides use of a non-natural sequence in preparation of an agent for detecting a target nucleic acid, where the non-natural sequence includes any one or more of the following: (1) a sequence containing both a deoxynucleotide and a ribonucleotide; (2) a sequence containing a deoxynucleotide and/or ribonucleotide, where the deoxynucleotide and/or ribonucleotide bears an artificially created modification that does not exist under natural conditions; and (3) a sequence containing a deoxynucleotide and/or ribonucleotide, where a backbone composed of the deoxynucleotide and/or ribonucleotide bears an artificially created modification that does not exist under natural conditions; and the agent further includes a Cas12 protein and crRNA.

In some specific embodiments, the non-natural sequence contains both a deoxynucleotide and a ribonucleotide.

In some specific embodiments, the deoxynucleotides and ribonucleotides in the non-natural sequence are arranged alternately at intervals. That is, a deoxynucleotide is followed by a ribonucleotide, or a ribonucleotide is followed by a deoxynucleotide.

In a further aspect, the present invention provides a probe, including a non-natural sequence, where the non-natural sequence includes any one or more of the following: (1) a sequence containing both a deoxynucleotide and a ribonucleotide; (2) a sequence containing a deoxynucleotide and/or ribonucleotide, where the deoxynucleotide and/or ribonucleotide bears an artificially created modification that does not exist under natural conditions; and (3) a sequence containing a deoxynucleotide and/or ribonucleotide, where a backbone composed of the deoxynucleotide and/or ribonucleotide bears an artificially created modification that does not exist under natural conditions. In some embodiments, the probe is capable of being trans-cleaved by the Cas12 protein. After the Cas12 protein binds to the target nucleic acid, the probe including the non-natural sequence is capable of being trans-cleaved by the Cas12 protein.

The probe refers to a probe with a fluorescent group introduced at one end of a non-natural sequence and a quenching group connected to the other end. The working principle of it is as follows: under normal circumstances, the fluorescence emitted by the fluorescent group is quenched by the quenching group due to the small distance between the fluorescent group and the quenching group. After the Cas protein cleaves the probe, the fluorescent group is separated from the quenching group, and the fluorescent group emits fluorescence normally, which is detected by an instrument, thereby achieving the purpose of detecting the target nucleic acid.

Further, the non-natural sequence in the probe is composed of nucleotides constituting RNA and nucleotides constituting DNA. The nucleotides constituting the RNA are any one or more of uracil ribonucleotide (rU), adenine ribonucleotide (rA), cytosine ribonucleotide (rC) and guanine ribonucleotide (rG), and the nucleotides constituting the DNA are any one or more of thymine deoxynucleotide (T), adenine deoxynucleotide (A), cytosine deoxynucleotide (C) and guanine deoxynucleotide (G).

Further, the probe is arranged in the order of ribonucleotide-deoxynucleotide or deoxynucleotide-ribonucleotide. The ribonucleotide is any one or more of a uracil ribonucleotide, an adenine ribonucleotide, a cytosine ribonucleotide and a guanine ribonucleotide, and the deoxynucleotide is any one or more of a thymine deoxynucleotide, an adenine deoxynucleotide, a cytosine deoxynucleotide and a guanine deoxynucleotide.

In some embodiments, the probe is 2-100 bp in length.

In some embodiments, the nucleotides are arranged sequentially in the order of rRD or DrR, where the rR is any one or more of rU, rA, rC and rG, and the D is any one or more of T, A, C and G.

It can be understood that according to the nucleotide arrangement rules of the non-natural sequence, it can be calculated that there are 4″ (n=the number of nucleotides in the non-natural sequence) types of non-natural sequences, e.g. a single chimera (poly ArA), a double chimera (poly rUArUA), a multiple chimera (UrACrGTrA), etc. Any non-natural sequence can be used in preparation of a probe to detect a target nucleic acid and is within the claimed scope of the present invention. Since the types of non-natural sequences are endless, the present invention designs 7 representative non-natural sequences and uses these 7 sequences for subsequent research.

Further, the probe sequence contains the following non-natural sequences: any one or more of rUArUArUA, ArUArUArU, rUrUrArUrUrU, TrUTrUTrU, ArAArAArA, CrCCrCCrC and GrGGrGGrG, where the rU is a uracil ribonucleotide, the A is an adenine deoxynucleotide, the Tis a thymine deoxynucleotide, the rA is an adenine ribonucleotide, the C is a cytosine deoxynucleotide, the rC is a cytosine ribonucleotide, the G is a guanine deoxynucleotide, and the rG is a guanine ribonucleotide.

Further, the probe sequence includes any one or more of rUArUArUA, ArUArUArU and TrUTrUTrU.

In the present invention, the commonly used LbCas12a protein and AsCas12a protein are selected to cleave the aforementioned 7 chimeric sequences respectively, and it has been found that both the two Cas12 proteins cannot cleave the GrGGrGGrG sequence. This may be because the spatial structure of the DNA or RNA sequence composed of guanine is relatively complex, or the interaction of the sequence itself and between the sequences is relatively large, which is not conducive to cleaving with the Cas12 protein; or it may be because the Cas12 family protein has a preference for not cleaving sequences composed of a large number of Gs. At the same time, the efficiency of both the LbCas12a protein and the AsCas12a protein in cleaving poly rUA and poly rAU can reach the same level as that of cleaving the TTATTT sequence (ssDNA), indicating that poly rUA and poly rAU are expected to become universal cleaving sequences of the Cas12a protein family. In addition, the effect of the LbCas12a protein in cleaving the TrUTrUTrU sequence is better than its effect in cleaving the ssDNA.

In still a further aspect, the present invention provides a system for detecting a target nucleic acid, including the aforementioned probe including a non-natural sequence, a Cas protein and crRNA. The crRNA can bind to the target nucleic acid and in turn activate the Cas protein. The activated Cas protein also non-specifically trans-cleaves the probe to achieve the effect of detecting the target nucleic acid, while cleaving the target sequence. The crRNA is designed according to the target nucleic acid sequence and the detection purpose.

Further, the Cas protein belongs to the Cas12 protein family.

Further, the Cas protein belongs to the Cas12a and Cas12b subfamilies.

Further, the Cas protein is any one or more of LbCas12a, AsCas12a and AapCas12b.

The present invention investigates the effect of chimeric sequences (rUArUArUA and ArUArUArU) on the trans-cleavage activity of Cas12a and Cas12b proteins. The results show that the enzyme kinetics of the LbCas12a and AsCas12a proteins when cleaving the chimeric sequences are equivalent to or even higher than those when cleaving an ssDNA. In general, the activity of the AapCas12b protein in trans-cleavage of the ssDNA, the ssRNA and the chimeric sequences is significantly weaker than those of the LbCas12a and AsCas12a proteins, but the effect of the AapCas12b in cleaving the chimeric sequences is better than those of conventional ssRNA sequences, and is more likely to cleave a poly ArU sequence.

In summary, compared with the conventional ssRNA, the chimeric sequences can improve the cleaving efficiency of the Cas12, and can even reach or exceed its effect in cleaving the ssDNA.

Further, when the Cas protein is LbCas12a, the effect is better when the probe sequence includes any one or more of rUArUArUA, ArUArUArU and TrUTrUTrU; and when the Cas protein is AsCas12a, the effect is better when the probe sequence includes any one or more of rUArUArUA and ArUArUArU.

Further, the target is DNA or RNA. When the target is RNA, the system also includes a DNA helper, which is a partial sequence or a simple PAM sequence bearing a PAM sequence and capable of binding to target RNA.

The Cas12a is firstly considered a DNA-targeting system. If the target sequence is RNA, it needs to be reverse-transcribed into cDNA before detection. The study in the present invention has shown that the introduction of a stretch of DNA helper bearing a PAM sequence enables the Cas12a system to effectively target RNA (see FIG. 4a for the specific principle), thereby eliminating the need to reverse-transcribe the RNA into the cDNA in advance.

In still yet a further aspect, the present invention provides a method for detecting a target nucleic acid, where the method conducts detection by using the system for detecting a target nucleic acid as described above, and the type of the target nucleic acid is DNA or RNA.

In some embodiments, the method includes the following steps: extracting DNA or RNA from a sample, and if the sample is RNA, it needs to reverse-transcribe the RNA into cDNA, or add a DNA helper to directly detect the RNA sample, where the DNA helper is a pair of DNA sequences which contain a PAM (protospacer adjacent motif) sequence, can be complementarily paired and form a stem-loop structure. A corresponding detection reaction system is prepared according to a detection object (DNA or RNA): 10 μL-20 μL of the aforementioned reaction solution is taken, and quickly transferred into a 96- or 384-well plate, and the plate is placed in a real-time fluorescence quantitative PCR instrument. Depending on the differences in the selected Cas12 protein, the reaction temperature of a Cas12a is 37° C.-42° C., while the reaction temperature of a high-temperature resistant Cas12 protein is 60° C.-65° C. The data is read once every 30 s-60 s, and the reaction time is 30 min-2 h. After the reaction is completed, a real-time fluorescence curve is generated or the final fluorescence value is obtained by measurement, and whether the target nucleic acid exists in the sample is determined according to the fluorescence curve or the fluorescence value. Three replicates and a blank control are set for each experiment. Pre-amplification of the sample can increase the detection sensitivity of the system.

In a further aspect, the present invention provides use of a non-natural sequence in preparation of an agent for preparing an agent for improving the efficiency of detecting target RNA. where the non-natural sequence is rUArUArUA, where the rU is a uracil ribonucleotide, and the A is an adenine deoxynucleotide. When the detection object is RNA, the detection effect of the Cas12 protein combined with the rUArUArUA sequence is the best, producing the highest signal-to-noise ratio, which has an effect better than that in the case when the detection object is ssDNA.

In a further aspect, the present invention provides use of a non-natural sequence in preparation of an agent for improving the trans-cleavage efficiency of a Cas12 protein, where the non-natural sequence is any one or more of rUArUArUA and TrUTrUTrU, where the rU is a uracil ribonucleotide, the A is an adenine deoxynucleotide, and the T is a thymine deoxynucleotide. Compared with conventional ssRNA sequences, the Cas12 has higher activity in cleaving chimeric sequences; and the efficiency of the LbCas12a protein in cleaving any one or more chimeric sequences of rUArUArUA and TrUTrUTrU is even higher than that in cleaving conventional ssDNA (TTATTT).

The beneficial effects of the present invention include:
1. the new use of the Cas12 protein in cleaving non-natural sequences and detecting nucleic acids (DNA and RNA) is discovered, and its detection effect is better than that in cleaving ssRNA, and in certain cases, it can be comparable to or even better than that of the conventional ssDNA probes, which significantly broadens the use of the Cas12 protein and the non-natural sequences;
2. the use of the non-natural sequences (chimeric sequences) is conducive to improving the detection effect of the Cas12 system, especially when the detection object is RNA, and it has also been proven that modifying the internal structure of the ssRNA can enhance the detection sensitivity of the ssRNA probes;
3. the preferences of different Cas12 proteins for trans-cleavaging probes are verified, that is, the same Cas12 protein has different efficiency in cleaving different probes, this property of the Cas12 protein can be utilized to design a multi-level detection scheme to achieve rich detection results with the simplest steps;
4. the LbCas12a and the AsCas12a can effectively cleave the chimeric sequences rUArUArUA and ArUArUArU, providing two universal probes that can be effectively cleaved by the Cas12a protein family for detection purposes;
5. the Cas12-chimeric sequence detection system is optimized, that is, the LbCas12a protein is suitable for use in combination with a variety of probes, among which the rUTrUTrU probe is the best, and the combined use of the AsCas12a protein with the chimeric sequences poly rUA and poly rAU is preferred;
6. provided is a method for detecting RNA without the need for a reverse transcription process, i.e., by adding a DNA helper;
7. pre-amplification of the detection sample (isothermal amplification or PCR) can improve the detection efficiency of the Cas12-chimeric probe system to the single-molecule level;
8. the Cas12-chimeric probe system can not only be combined with microfluidics technology for high-throughput detection, but also be used for clinical testing;
9. it further deepens researchers' understanding of the Cas12 and the non-natural sequences, providing new directions for the optimization of the CRISPR/Cas12 detection system; and
10. there are no nucleases in nature that can degrade non-natural sequences, so the half-lives of the non-natural sequences are longer than those of the DNA and RNA, and thus probes made from the non-natural sequences can be preserved for a longer time; and the detection stability of non-natural sequence probes is higher, and the detection results are more reliable.

### BRIEF DESCRIPTION OF THE DRAWINGS

To describe the technical solutions in the embodiments of the present invention or in the prior art more clearly, the following briefly describes the accompanying drawings required for describing the embodiments or the prior art. Apparently, the accompanying drawings in the following description show some embodiments of the present invention, and a person of ordinary skill in the art may still derive other drawings from these accompanying drawings without creative efforts.

FIG. 1a shows a schematic diagram of a workflow of a Cas12 protein trans-cleavage system, where ssDNA (TTATT), ssRNA (poly rU and poly rA), an RNA motif sequence (T*A*rArU*G*C) and a chimeric sequence (ArUArUArU and rUArUArUA) are all labeled with fluorescent groups and quenching groups at the same time to make probes, which are applied to a Cas12 protein detection system;

FIG. 1b shows the ability of a LbCas12a protein in an activated state to trans-cleave an ssDNA probe, where NTC represents a non-template control, which is a negative control, an error bar represents standard deviation, and n=3;

FIGS. 1c-e show the abilities of the LbCas12a protein in the activated state to trans-cleave ssRNA (rUrUrUrUrUrUrU, rArArArArArA and T*A*rArU*G*C) probes; where NTC represents a non-template control, which is a negative control, an error bar represents standard deviation, and n=3;

FIGS. 1f-g show the abilities of the LbCas12a protein in the activated state to trans-cleave chimeric probes (rUArUArUA and ArUArUArU) based on ssRNA (poly rU) modifications; where NTC represents a non-template control, which is a negative control, an error bar represents standard deviation, and n=3;

FIGS. 2a-d show the abilities of the LbCas12a protein in the activated state to trans-cleave conventional sequences, monomeric ssDNA, monomeric ssRNA and chimeric sequences; where NTC represents a non-template control, which is a negative control, an error bar represents standard deviation, and n=3;

FIGS. 2e-h show the abilities of the AsCas12a protein in the activated state to trans-cleave conventional sequences, monomeric ssDNA, monomeric ssRNA and chimeric sequences; where NTC represents a non-template control, which is a negative control, an error bar represents standard deviation, and n=3;

FIGS. 3a-b show the effects of chimeric sequences on the trans-cleavage activity of the LbCas12a protein; where NTC represents a non-template control, which is a negative control, an error bar represents standard deviation, n=3; and the concentration of an activator of the LbCas12a protein in the detection system in FIG. 3a is 10 nM;

FIGS. 3c-d show the effects of chimeric sequences on the trans-cleavage activity of the AsCas12a protein; where NTC represents a non-template control, which is a negative control, an error bar represents standard deviation, n=3; and the concentration of an activator of the AsCas12a protein in the detection system in FIG. 3c is 10 nM;

FIGS. 3e-f show the effects of chimeric sequences on the trans-cleavage activity of the AapCas12b protein; where NTC represents a non-template control, which is a negative control, an error bar represents standard deviation, n=3; and the concentration of an activator of the AapCas12b protein in the detection system in FIG. 3e is 10 nM;

FIG. 4a shows a schematic diagram of the principle of the Cas12a system in detecting RNA;

FIG. 4b shows a real-time reaction curve of the AsCas12a system in detecting RNA; where NTC represents a non-template control, which is a negative control, an error bar represents standard deviation, n=3;

FIGS. 5a-b show a flow chart of detecting samples with pre-amplification/without amplification, where the probe used in FIG. 5a is ssDNA and the probe used in FIG. 5b is a chimeric sequence;

FIGS. 5c-d show the detection effects of a pre-amplification/no amplification-LbCas12a-ssDNA/chimeric sequence system; where NTC represents a non-template control, which is a negative control, an error bar represents standard deviation, and n=3;

FIGS. 5e-f show the detection effects of a pre-amplification/no amplification-AsCas12a-ssDNA/chimeric sequence system; where NTC represents a non-template control, which is a negative control, an error bar represents standard deviation, and n=3;

FIG. 6a shows a schematic diagram of a workflow of microdroplet microfluidics technology;

FIG. 6b shows a detection effect of a Cas12-chimeric probe system combined with a microfluidic chip;

FIG. 6c shows a flow chart of the Cas12-chimeric probe system for detecting COVID-19 clinical saliva samples;

FIG. 6d shows results of detecting COVID-19 by the Cas12-chimeric probe system;

FIG. 7a shows a flow chart of RAPID detection employing different probes and different Cas12 proteins;

FIG. 7b shows RAPID performance detection results using AsCas12a and different probes; and

FIG. 7c shows RAPID performance detection results using LbCas12a and different probes.

### DETAILED DESCRIPTION OF THE INVENTION

The present invention is further described in detail hereafter in conjunction with the accompanying drawings and specific embodiments. The embodiments are only used for explaining the present invention, and are not intended to limit the scope of the present invention. The LbCas12a and mouse RNase inhibitor used in the following examples are purchased from New England Biolab (NEB), the AsCas12a protein is purchased from IDT, and the AapCas12b (addgene #153162) is obtained by purifying according to the previously published article [15]. All probes, crRNA and primers as used are synthesized by IDT, and the probe bears a 56-FAM group at the 5′ terminus thereof and a 3IABKFQ group at the 3′ terminus thereof. The activators are all available from IDT. Unless otherwise specified, the remaining materials, agents, and the like as used are commercially available. Unless otherwise specified, the test methods as used are all conventional methods. Three replicates are set when the fluorescence value is determined.

### Example 1: A Non-DNA Sequence could be Trans-Cleaved by a LbCas12a Protein

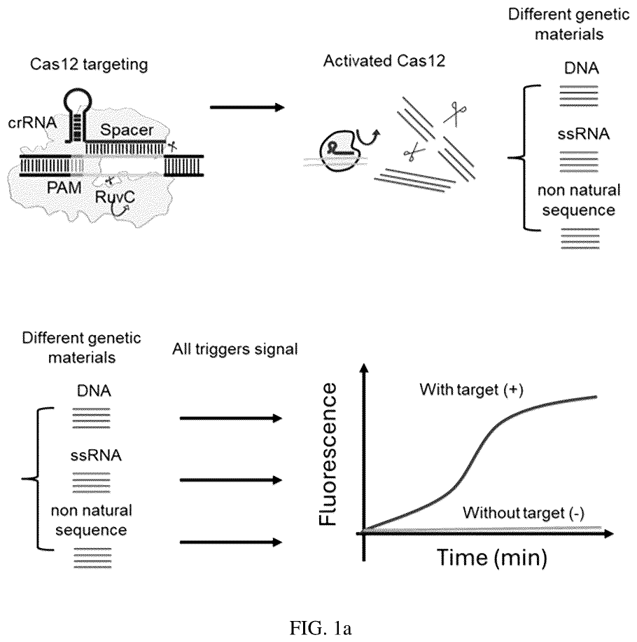

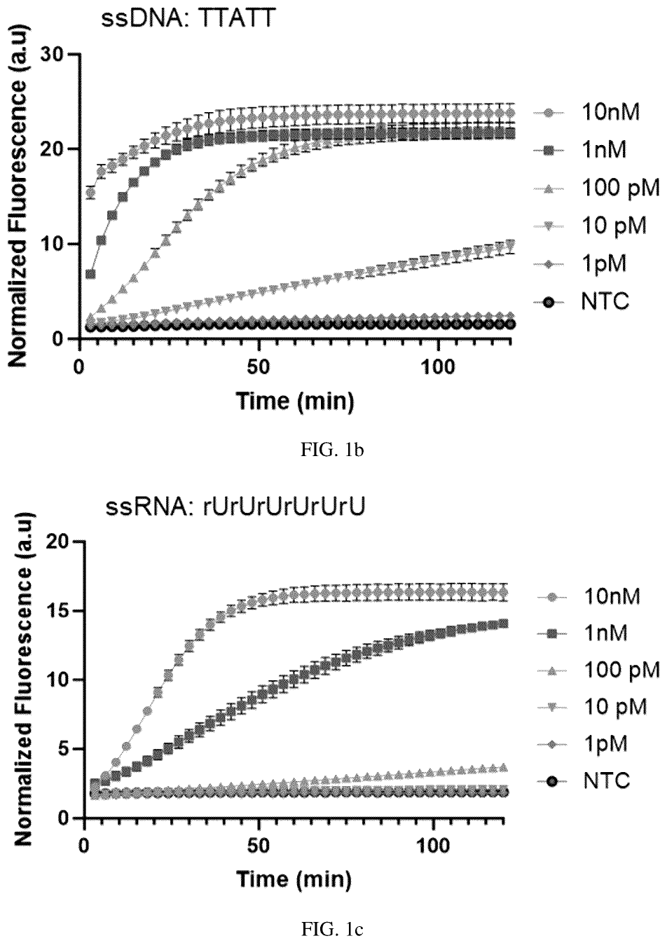

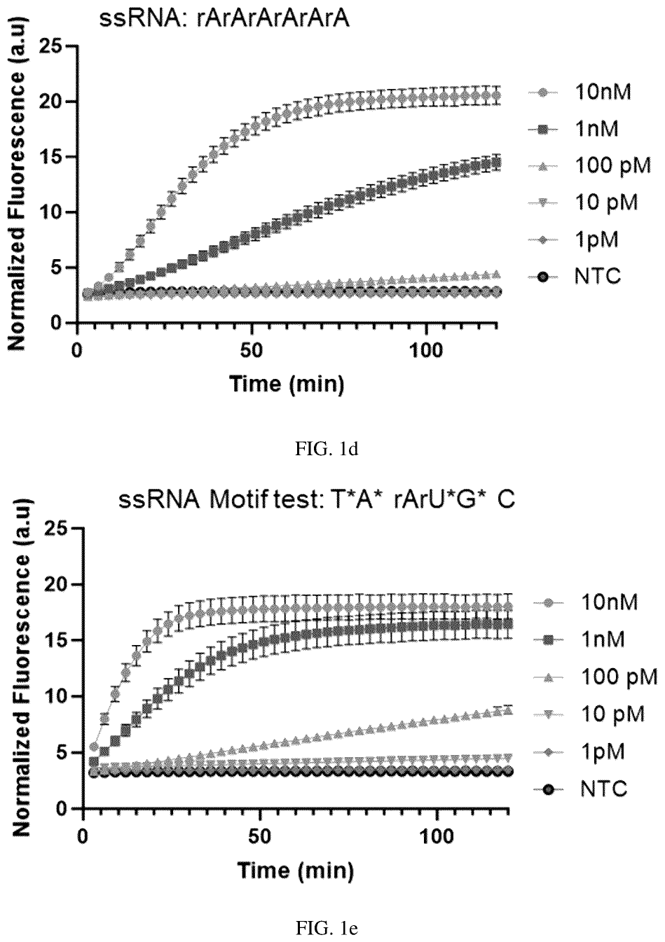

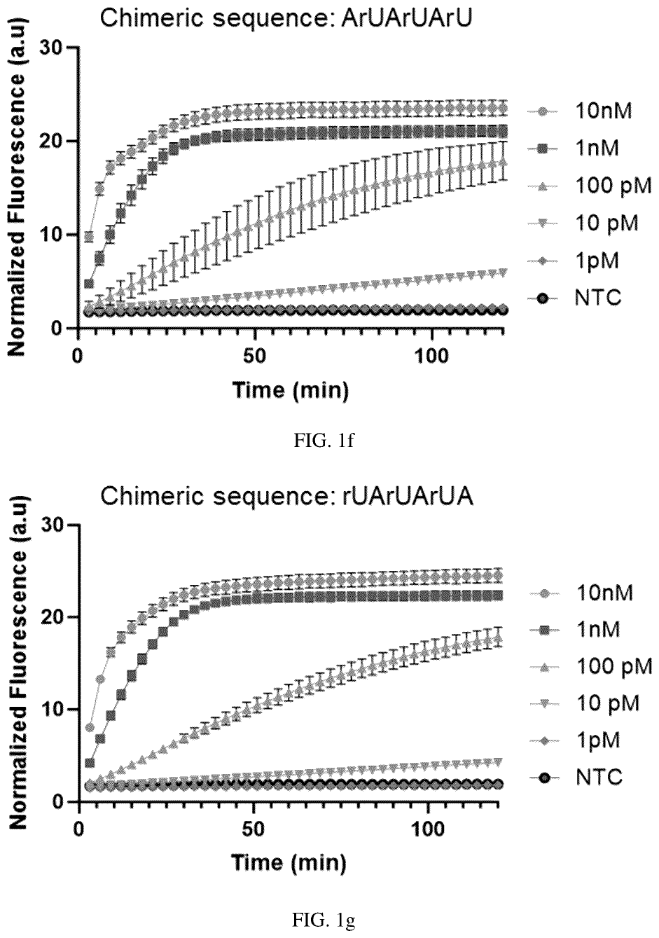

In order to study the ability of a Cas12 protein to trans-cleave a non-DNA sequence, this example used a LbCas12a protein to cleave ssDNA, ssRNA, RNA dinucleotide sequences (sequences containing two ribonucleotides) and non-natural chimeric sequences, with 3 replicates set for each experimental group. The experimental principle of this example is shown in FIG. 1a. Under the guidance of crRNA, the Cas12 protein identified a target DNA nucleic acid sequence (activator), and then its trans-cleavage activity was activated to randomly cleave an adjacent nucleic acid probe, one end of which had a fluorescent group (preferably a FAM group in this example) and the other end of which had a quenching group (preferably a 3IABkFQ group in this example). The fluorescence of the cleaved probe could be excited, which could be identified by a machine, thereby achieving the purpose of detecting the target nucleic acid. It should be understood that the crRNA sequence in the Cas12 detection system could be replaced according to different detection objects to meet different detection requirements; and at the same time, when the content of the target DNA (activator) was higher, more Cas12 protein would be activated faster, more probes would be cleaved faster, the emitted fluorescence would be stronger, and the detection results would be more accurate.

Specifically, this example employed a partial HPV 18 sequence (SEQ ID NO: 1) as an activator of Cas12, which was diluted into different gradients (10 nM, 1 nM, 100 pM, 10 pM and 1 pM) to further explore the effects of different probes on the detection sensitivity of the Cas12 system. Three replicates were set for each experiment. The HPV 18 nucleic acid was the same as that described in the article [16]. The probe was ssDNA (TTATT) (SEQ ID NO: 2), SSRNA (poly rU (SEQ ID NO: 3) and poly rA (SEQ ID NO: 4), and a non-natural sequence (T*A*rArU*G*C (SEQ ID NO: 5, * represented a phosphorothioate bond modification), poly ArU (SEQ ID NO: 6) and poly rUA (SEQ ID NO: 7)) with a FAM fluorescent group and a 3IABkFQ quenching group, which acted as a reporter element in the Cas12 detection system. The TTATT (T) was known ssDNA that could be effectively cleaved by the Cas12 protein. In all the examples of the present invention, this sequence served as a positive control. The specific experimental operations were as follows: firstly 18 μL of a master mix without the Cas12 activator was formulated, where the components in the system and final concentrations thereof were as follows: a 1×NEB 2.1 buffer (New England Biolab), 50 nM of Cas12 (the type of the Cas12 protein in this example was LbCas12a), 50 nM of crRNA (the specific sequence was shown in SEQ ID NO: 8), 10 U of a rat RNase inhibitor (RNase inhibitor Murine) and 125 nM of a probe, specifically, 2 μL of a NEB 2.1 buffer (10×), 0.5 μL of 2 μM LbCas12a, 0.5 μL of 2 μM crRNA (the specific sequence was as shown in SEQ ID NO: 8), 0.25 μL of a 40 U/μL rat RNase inhibitor (RNase inhibitor Murine) and 0.5 μL of a 5 μM probe; subsequently, 2 μL of the Cas12 activator (partial synthetic sequence of HPV 18, SEQ ID NO: 1) was added into the master mix, while in a negative control group the added activator was replaced with 2 μL of ddH₂O, and then they were mixed evenly. The Cas12 activator was diluted to final reaction concentrations of 10 nM, 1 nM. 100 pM, 10 pM and 1 pM, respectively; and then 10 μL of the aforementioned reaction solution was taken and quickly transferred into a 384-well plate, and the fluorescence generated by each reaction system every minute was determined by a real-time fluorescence quantitative PCR instrument (Roche LightCycler 480 II) at 37° C. for 2 hours to generate a real-time fluorescence curve, with 3 replicates set for each reaction system.

As shown in FIG. 1b, within the reaction time of 1 hour, when the concentration of HPV 18 was 10 pM, the fluorescence generated by the ssDNA probe could still be detected by an instrument. This might be because the DNase activity of LbCas12a was very strong and the intrinsic limit of detection of LbCas12a was at a pM level. As long as a small part of the LbCas12a protein was activated, the intensity of the fluorescence emitted by the probe cleaved by it could reach a detectable intensity. On the other hand, this result indicated that when the ssDNA was used as a probe, the sensitivity of the LbCas12a detection system could reach 10 pM; and in contrast, under the same conditions, when the detection system used ssRNA (rUrUrUrUrUrU or rArArArArArA) as a probe, the sensitivity can only reach the nM level (FIGS. 1c and 1d). In addition, these experimental results indicated that the activated LbCas12a protein has the ability to trans-cleave the ssDNA and the SSRNA.

Next, in order to explore whether activated LbCas12a could non-specifically cleave the RNA motif sequence, the activated LbCas12a protein was used to cleave the RNA motif sequence (T*A*rArU*G*C. * represented phosphorothioate bond modification) in this example, and this experiment was repeated 3 times. The T*A*rArU*G*C sequence can be understood as a sequence formed by adding modifications of phosphorothioate bonds and deoxyribonucleotides on the basis of the sequence composed of two ribonucleotides, i.e. rA and rU, and the phosphorothioate bond plays a role in preventing the sequence from being cleaved.

The results are shown in FIG. 1e. Compared with the probes composed of only one type of ribonucleotide (rU or rA), the sequence probes composed of two types of ribonucleotides could increase the sensitivity of the Cas12 detection system by at least 10 times. Further, its trans-cleavage ability was affected by the nature of the sequence itself. Therefore, the identification of the trans-cleavage activity of Cas12 should not only be limited to specific sequence types. Instead, the scope of exploration objects to bases and other structures inside nucleic acids.

Based on the above conclusion, two non-natural sequences, i.e. rUArUArUA (poly rUA) and ArUArUArU (poly ArU), were designed in this example, where A the adenine deoxyribonucleotide that constitutes DNA, and rU represented the uracil ribonucleotide that constitutes RNA. Three replicates were set for each experimental group. It was worth noting that these two sequences were formed by introducing deoxyribonucleotides on the basis of RNA sequence synthesis. Since deoxyribonucleotides were the constituent units of DNA, these sequences were also called chimeric sequences or non-natural sequences. Unexpectedly, the limit of detection (LOD) of these chimeric sequences was higher than that of the aforementioned RNA motif sequences (T*A*rArU*G*C), and even reached a level similar to that of ssDNA probes (FIGS. 1f and 1g).

In summary, LbCas12a could non-specifically cleave ssDNA and ssRNA. Under normal circumstances, its activity of cleaving ssDNA was significantly higher than that of cleaving ssRNA (rUrUrUrUrUrU, rArArArArArA or T*A*rArU*G*C, FIGS. 1c and 1d). However, through the modification of ssRNA sequences, such as the introduction of deoxyribonucleotides (poly rUA or poly ArU), the gap or nick between the activity of LbCas12a in cleaving ssDNA and that in cleaving ssRNA could be narrowed (FIGS. 1e and 1g). It should be noted that the modified ssRNA sequences were all non-natural nucleic acid sequences.

### Example 2: Preferences of LbCas12a and AsCas12a in Trans-Cleavage of Sequences

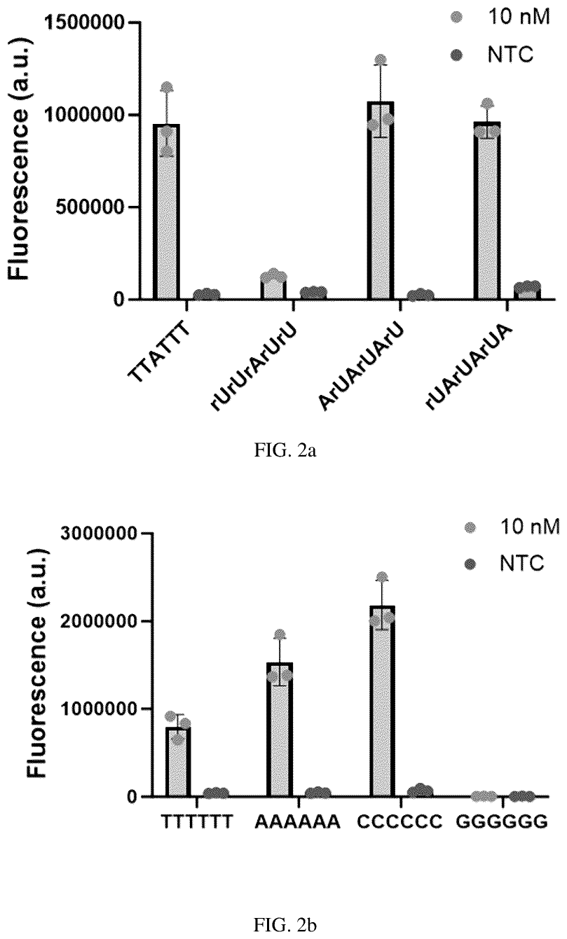

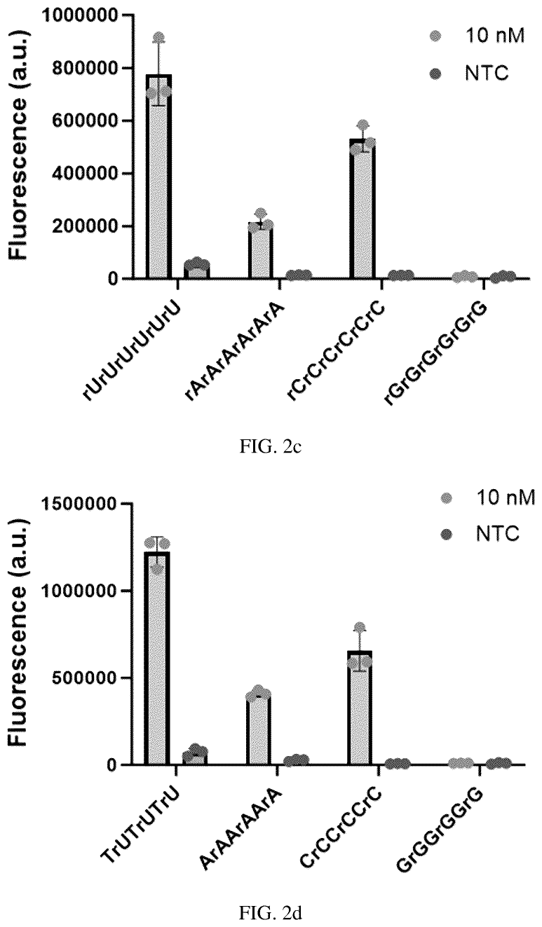

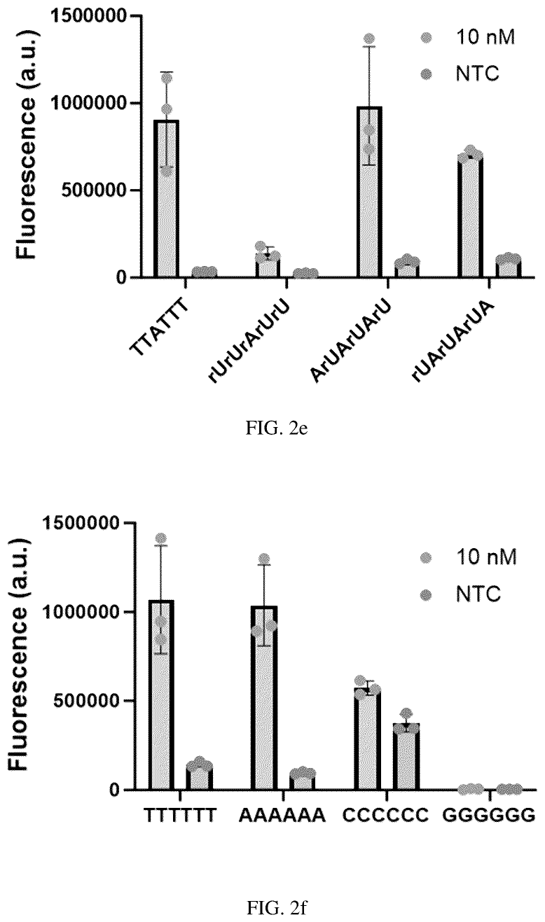

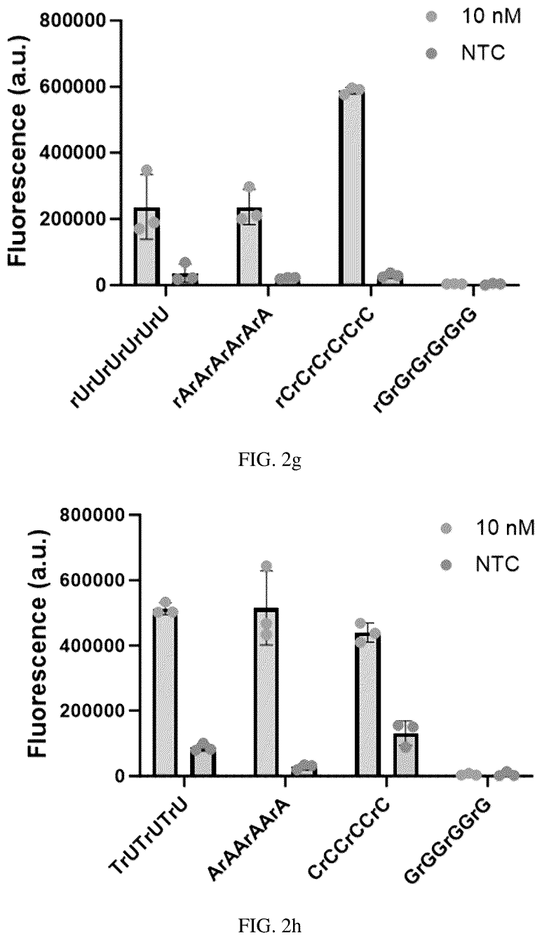

The nuclease activity of LbCas12a (which can cleave both ssDNA and ssRNA) implies that the cleavage mechanisms of other members of the Cas12 protein family are also independent of sequence types (such as DNA or RNA). Therefore, two commonly used Cas12 proteins (LbCas12a and AsCas12a) were selected in this example to explore their sequence preference. In this example, 16 different single-stranded nucleic acid sequences were designed, including ssDNA sequences (TTATTT (SEQ ID NO: 9), AAAAAA (SEQ ID NO: 10), GGGGGG (SEQ ID NO: 11), CCCCCC (SEQ ID NO: 12) and TTTTTT (SEQ ID NO: 13)), ssRNA sequences (rArArArArArA (SEQ ID NO: 4), rGrGrGrGrGrG (SEQ ID NO: 14), rCrCrCrCrCrC (SEQ ID NO: 15), rUrUrUrUrUrU (SEQ ID NO: 3)) and chimeric sequences (poly rUA (SEQ ID NO: 7), poly ArU (SEQ ID NO: 6), rUrUrArUrUrU (SEQ ID NO: 16), TrUTrUTrU (SEQ ID NO: 17), ArAArAArA (SEQ ID NO: 18), CrCCrCCrC (SEQ ID NO: 19) and GrGGrGGrG (SEQ ID NO: 20)), and the TTATTT was a known ssDNA sequence that could be effectively cleaved by the Cas12 protein. In addition, a reaction system with a total volume of 20 μL was constructed. The reaction system and procedure were the same as those described in Example 1. The final reaction concentration of the Cas12 activator was 10 nM. The reaction was performed at 37° C. for 2 h, and its final fluorescence value was determined using a real-time fluorescence quantitative PCR instrument QuantStudio 5 real-time PCR system (Thermo Fisher Scientific). The specific experimental results are shown in FIG. 2 and Table 1.

It was worth noting that when cleaving the same probe, there were differences in the cleaving abilities of LbCas12a protein and AsCas12a protein. However, the sequences that could be cleaved by the two were roughly the same. In other words, the probes that could be cleaved by LbCas12a protein could also be cleaved by AsCas12a protein, and vice versa. The efficiency of the two Cas12 proteins in cleaving the chimeric sequences of poly rUA and poly rAU could reach the level of cleaving the TTATTT sequence (FIGS. 2a and 2e). However, the signal-to-noise ratio generated when cleaving the rUrUrArUrUrU sequence was very low (FIGS. 2a and 2e, Table 1). This implied that the two Cas12 proteins had no preference for trans-cleavage of the RNA sequence in which a single rU in the poly rU sequence was replaced by rA. This modification method of the RNA sequence even caused its signal-to-noise ratio intensity to be lower than that of both poly rU and poly rA (FIGS. 2c and 2g).

When the probe type was monomeric ssDNA (FIGS. 2b and 2f, Table 1), the fluorescence value generated by the LbCas12a protein cleaving the CCCCCC sequence was the highest, followed by that of the AAAAAA sequence. The AsCas12a protein had relatively strong abilities to cleave the TTTTTT sequence and the AAAAAA sequence. However, both of these two proteins could hardly cleave the GGGGGG sequence. When cleaving the rGrGrGrGrGrG sequence which belongs to the ssRNA type and the chimeric sequence GrGGrGGrG, the same phenomenon occurred, indicating that neither of the two Cas12 proteins could cleave the nucleotide sequences composed of guanine, regardless of whether the guanine was connected to a deoxyribose or ribose. This may be because the above 3 poly (r)G sequences themselves or among them could form relatively complex and stable spatial structures, which were not conducive to the cleavage by the Cas12 proteins. Alternatively, these sequences tended to bind to the repetitive sequences on the chromosome (chromatin), and these repetitive sequences were relatively far away from the Cas12 proteins that recognized and bound to the target sequences. Since Cas12 could only cleave the adjacent sequences non-specifically, the poly (r)G sequences could not be cleaved by the Cas12 proteins. In addition, when cleaving ssDNA sequences, the background fluorescence generated by the AsCas12a system was higher than that of LbCas12a, which was consistent with previous reports [17]. This may be because AsCas12a had a certain trans-cleavage activity when it was not guided by crRNA to recognize the target sequence, and this activity was stronger than that of LbCas12a. When the probe type was monomeric ssRNA (FIGS. 2c and 2g, Table 1), the LbCas12a protein was most suitable for cleaving the rUrUrUrUrUrU sequence, while AsCas12a was suitable for cleaving the rCrCrCrCrCrC sequence. Introducing ribonucleotides on the basis of ssRNA could improve the cleavage efficiency of the Cas12 protein to a certain extent (FIGS. 2d and h, Table 1). For example, on the basis of the rUrUrUrUrUrU sequence, thymidine deoxyribonucleotide was introduced to prepare the TrUTrUTrU sequence. The effect of LbCas12a in cleaving the latter was far better than that of the former, and even better than the effect of cleaving the TTATTT sequence. Meanwhile, modifying the rUrUrUrUrUrU sequence into the TrUTrUTrU sequence or modifying the rArArArArArA sequence into the ArAArAArA sequence could also improve the cleavage effect of AsCas12a.

TABLE 1
Effects of different Cas12 proteins on trans-
cleavage of different nucleic acid sequences

| | | | | Signal-to-noise |
|---|---|---|---|---|
| | | | | ratio |
| | | | | (fluorescence |
| | | | | value generated |
| Sequence | Specific | Type of Cas12 | Fluorescence | by cleaving the |
| Type | sequence | protein | value (a.u.) | probe/NTC) |
| ssDNA | TTATTT | LbCas12a | <1,000,000 | High |
| | or TTATT | AsCas12a | <1,000,000 | High |
| | AAAAAA | LbCas12a | ≈1,500,000 | High |
| | | AsCas12a | ≈1,000,000 | High |
| | GGGGGG | LbCas12a | 0 | 0 |
| | | AsCas12a | 0 | 0 |
| | CCCCCC | LbCas12a | >2,000,000 | High |
| | | AsCas12a | ≈500,000 | Lower |
| | TTTTTT | LbCas12a | <1,000,000 | High |
| | | AsCas12a | >1,000,000 | High |
| ssRNA | rArArArArArA | LbCas12a | ≈200,000 | High |
| | | AsCas12a | >200,000 | High |
| | rGrGrGrGrGrG | LbCas12a | 0 | 0 |
| | | AsCas12a | 0 | 0 |
| | rCrCrCrCrCrC | LbCas12a | <600,000 | High |
| | | AsCas12a | <600,000 | High |
| | rUrUrUrUrUrU | LbCas12a | <800,000 | High |
| | | AsCas12a | >200,000 | High |
| Chimeric | rUArUArUA | AsCas12a | >1,000,000 | High |
| sequence | (polyrUA) | LbCas12a | <1,000,000 | High |
| | ArUArUArU | AsCas12a | <1,000,000 | High |
| | (polyrAU) | LbCas12a | ≈750,000 | High |
| | rUrUrArUrUrU | AsCas12a | <250,000 | Low |
| | | LbCas12a | <250,000 | Low |
| | TrUTrUTrU | AsCas12a | <1,500,000 | High |
| | | LbCas12a | ≈500,000 | High |
| | ArAArAArA | AsCas12a | <500,000 | High |
| | | LbCas12a | ≈500,000 | High |
| | CrCCrCCrC | AsCas12a | <1,000,000 | High |
| | | LbCas12a | >400,000 | High |
| | GrGGrGGrG | AsCas12a | 0 | 0 |
| | | LbCas12a | 0 | 0 |

In summary, both LbCas12a protein and AsCas12a protein had the ability to cleave chimeric sequences (except for GrGGrGGrG), and when cleaving certain chimeric sequences, their trans-cleavage activities were equivalent to or even better than the activity of cleaving TTATTT. Further, when applying LbCas12a and AsCas12a proteins to the field of nucleic acid detection, the sequences of chimeric probes can be selected according to their preferences for trans-cleavage sequences [18], and conversely, suitable Cas12 proteins can also be selected based on the sequences of chimeric probes. In this design concept, different variants of the Cas12 protein could exhibit limited trans-cleavage activities on certain sequences, but showed high enzyme kinetics on specific sequences. Next, by combining different Cas12 variants with specific probe materials, each combination was used to detect different target nucleic acid sequences. In the reaction, the recognition of specific probes corresponded to the activation of specific Cas proteins, which further indicated that the special crRNA recognized a certain sequence, allowing different targets to be identified. See Table 2 for details, where V represents a better choice, and x represents not applicable. It can be seen from Table 2 that, compared with the AsCas12a protein, the LbCas12a protein had a wider range of applications. On the other hand, both the chimeric sequences poly rUA and poly rAU could be effectively cleaved by the two proteins described or could be used as universal chimeric nucleic acid probes. Further, when the cleavage object was a chimeric sequence, the LbCas12a protein was most suitable to be used in combination with the TrUTrUTrU probe, followed by the poly rUA probe and the poly rAU probe, and the AsCas12a protein was better when combined with the chimeric sequences poly rUA and poly rAU. Overall, the research results of this example highlighted the sequence preference of LbCas12a and AsCas12a in trans-cleavage reactions, as reflected in the type of genetic materials or specific sequences. Understanding these preferences is critical for designing and developing multi-level one-to-one diagnosis using Cas12 family proteins.

TABLE 2
Preferences of LbCas12a and AsCas12a in
trans-cleavage of nucleic acid sequences

| | | | rUr | | | | |
|---|---|---|---|---|---|---|---|
| | | | UrA | TrU | ArA | CrC | GrG |
| | poly | poly | rUr | TrU | ArA | CrC | GrG |
| | rUA | rAU | UrU | TrU | ArA | CrC | GrG |
| LbCas12a | √ | √ | x | √ | | √ | x |
| AsCas12a | √ | √ | x | | √ | | x |

### Example 3: Effect of Chimeric Sequences on Trans-Cleavage Activities of Cas12a and Cas12b Proteins

In order to explore the influence of combination with non-ssDNA probes on the limit of detection (LOD) of different Cas12 proteins, in this example, LbCas12a, AsCas12a and AapCas12b were taken as the research objects, and 5 different types of probes were selected, including one ssDNA probe (TTATT, SEQ ID NO: 2), 2 ssRNA probes (rUrUrUrUrUrU (SEQ ID NO: 3) and rArArArArArA (SEQ ID NO: 4)) and two chimeric probes (rUArUArUA (SEQ ID NO: 7) and ArUArUArU (SEQ ID NO: 6)), and Cas protein was activated with different concentrations (1 nM, 100 pM and 10 pM) of activator. The 3 proteins represent two subfamilies of Cas12 proteins (Cas12a and Cas12b protein families) and are commonly used in the field of diagnosis and gene editing [15, 19]. Cas12a protein can undergo enzymatic reactions at room temperature (37° C.), while AapCas12b is a thermostable protein that functions at 60° C. Therefore, the type of Cas12 can be selected by temperature conditions, and then combined with its preferred chimeric probe.

In this example, the Cas12 protein activator adopted was the post-transcriptional DNA sequence of the N-terminal partial sequence of the COVID-19 gene (SEQ ID NO: 21), and the crRNA sequence was the same as that described in SEQ ID NO: 22. The remaining components and detection steps of the LbCas12a and AsCas12a detection systems were the same as those described in Examples 1-2. The steps of the AapCas12b cleavage experiment were as follows: a reaction system was prepared with a total volume of 20 μL, where the components of the system and their final concentrations were as follows: 1× isothermal amplification buffer (LAMP, NEB), 100 nM AapCas12b protein, 100 nM crRNA (the specific sequence was as shown in SEQ ID NO: 22), 125 nM probe and 2 μL DNA activator (SEQ ID NO: 21), the reaction was performed at 60° C. for 2 h. and the remaining steps were the same as those described in Examples 1-2.

Existing studies have demonstrated that utilizing the Cas12a protein to trans-cleave ssDNA probes can be used to detect target nucleic acids at the picomolar level [6, 20]. Hence, in this example, the ssDNA (TTATT) probe was adopted as a positive control.

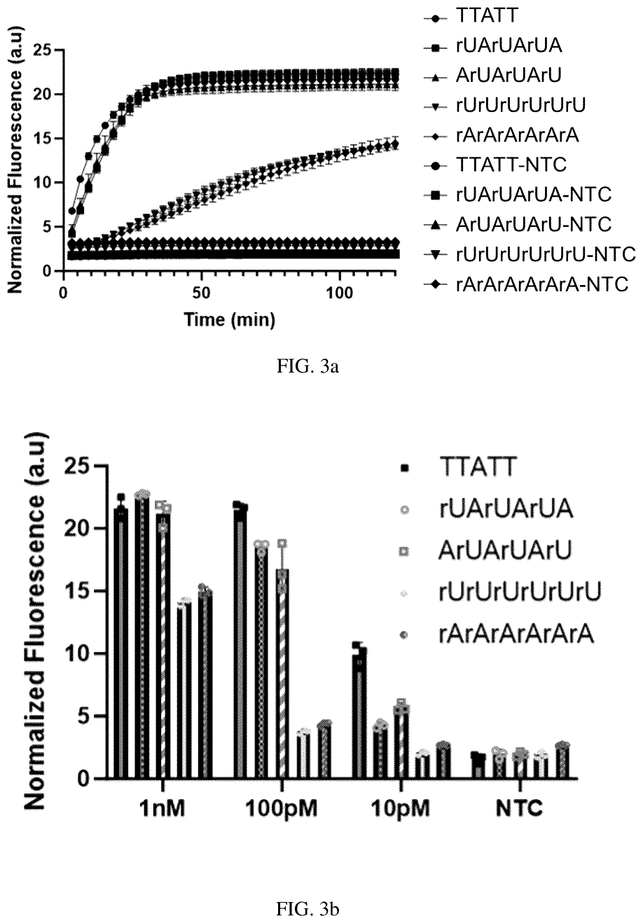

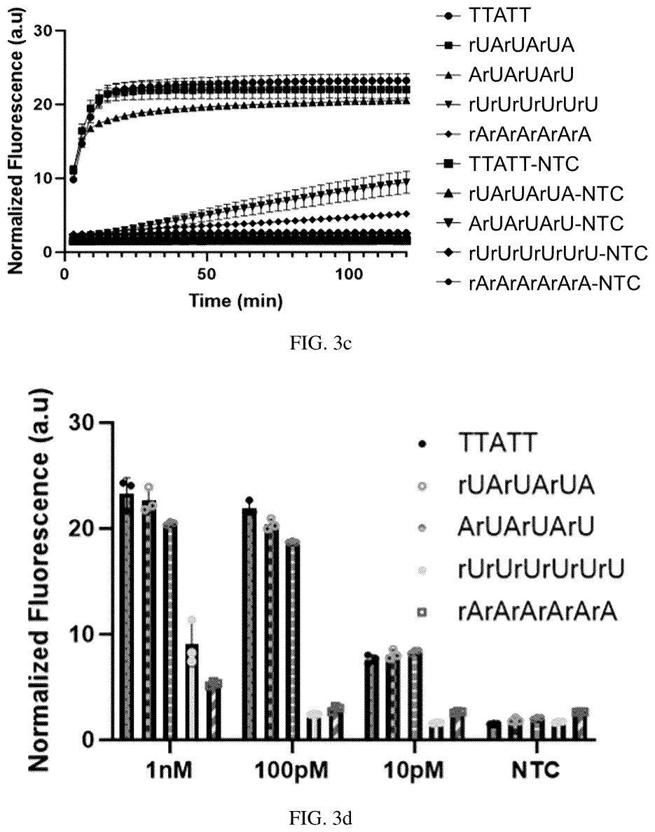

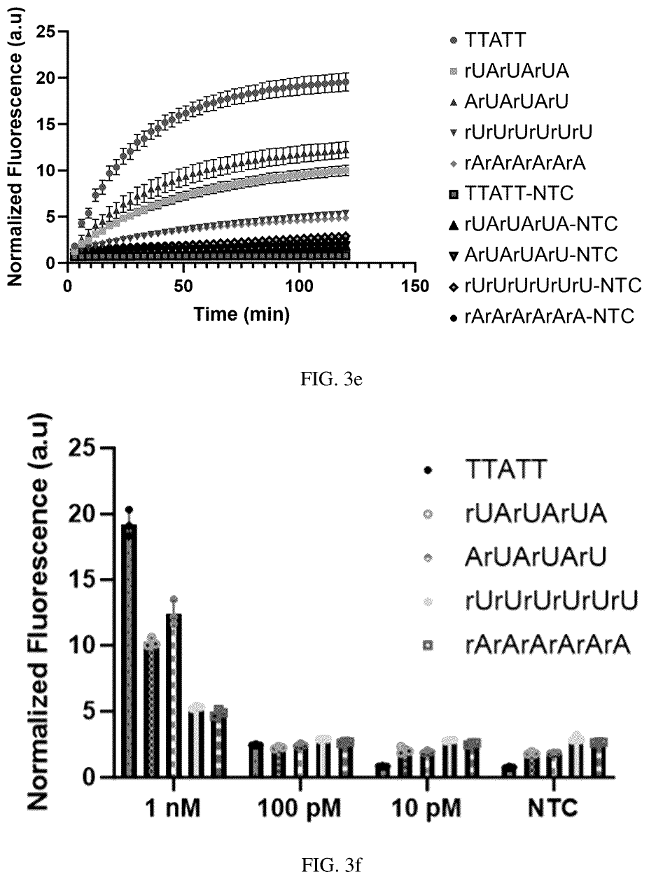

Consistent with the experimental results of Example 2, the enzymatic kinetics of the LbCas12a protein and the AsCas12a protein when cleaving chimeric sequences were comparable to or even higher than those when cleaving ssDNA (FIGS. 3a-d). In addition, the use of chimeric probes could also enable the detection limits of the above two proteins to reach the picomolar level. It was worth noting that AsCas12a exhibited stronger enzymatic kinetics than LbCas12a, which meant that it took a shorter time to reach the signal peak (FIGS. 3a and 3c). Conversely, the trans-cleavage activity of the AapCas12b protein was significantly weaker than that of the two aforementioned Cas12a proteins (FIGS. 3e-f). First, the AapCas12b protein exhibited a slower reaction rate, which meant that it took at least 60 minutes to reach the signal peak. Second, the RNase activity of the AapCas12b protein was weaker. Third, the background fluorescence generated during the cleavage by AapCas12b was stronger. It was worth noting that the effect of AapCas12b in cleaving chimeric sequences was better than that of conventional ssRNA sequences. Further, AapCas12b was more inclined to cleave the poly ArU sequence rather than the poly rUA sequence, which was contrary to the preference of the Cas12a proteins.

Overall, compared with the conventional ssRNA, the chimeric sequences could improve the cleaving efficiency of the Cas12, and could even reach or exceed its effect in cleaving the ssDNA.

### Example 4: Effect of Detection of RNA Using AsCas12a and Non-Natural Sequences

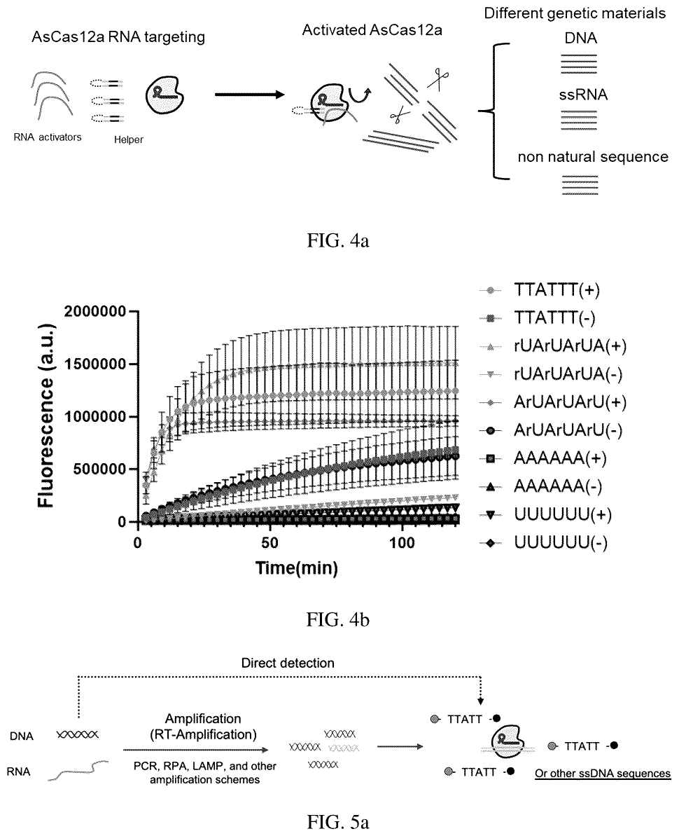

The Cas12a is firstly considered a DNA-targeting system. If the target sequence is RNA, it needs to be reverse-transcribed into cDNA before detection. However, our latest research has shown that the introduction of a DNA helper with a PAM sequence can enable the Cas12a system to effectively target RNA (see FIG. 4a for the specific principle). Therefore, in this example, the recognition ability of the above system for target RNA was optimized by screening the types of reverse cleavage sequences. The types of the reverse cleavage sequences were the same as those described in Example 3 ( ). The concentrations of the target RNA were all 50 nM, and the RNA sequence was shown as in SEQ ID NO: 23. Three replicates were set for each experimental group. The specific experimental results were as follows: a 50 μL reaction system was prepared, where the components of the system and their final concentrations were as follows: 1×NEB 2.1 buffer, 90 nM AsCas12a, 50 U RNase inhibitor, 90 nM crRNA (the specific sequence was the same as that shown in SEQ ID NO: 24), 20 nM DNA helper (the helper sequences were as shown in SEQ ID NO: 25 and SEQ ID NO: 26), 31.25 mM MgCl2, 500 nM probe and 50 nM RNA activator (the specific sequence was the same as that shown in SEQ ID NO: 23); 15 μL of the above reaction solution was taken in a 384-well plate, and 3 replicates were set; and the fluorescence intensity was measured using a real-time fluorescence quantitative instrument (QuantStudio 5 real-time PCR system ThermoFisher Scientific) once per minute at 37° C. for 2 h, and then a real-time fluorescence curve was generated and the final fluorescence value was obtained through detection.

Overall, all the probes could be cleaved and then trigger fluorescence signals, which confirmed that the DNA helper could indeed assist the Cas12a system to directly detect RNA without the need to first reverse-transcribe RNA into cDNA before conducting the detection. However, among the 5 types of probes, the chimeric sequence poly rUA exhibited the best detection effect, which meant that it produced the highest signal-to-noise ratio that was better than that of ssDNA (FIG. 4b). Unexpectedly, compared with the effect of detecting DNA, monomeric ssRNA (poly rU and poly rA) showed a rather weak RNA detection ability, which indicated that although the auxiliary method could enable the Cas12 protein to directly recognize RNA and activate its trans-cleavage activity, the trans-cleavage activity of the Cas12 protein activated by RNA was weaker than that activated by DNA. In conclusion, combined with the results of Examples 2 and 3, this example further demonstrated that the chimeric sequence could improve the detection effect of the Cas12 system, and further expanded the application fields of the Cas12 protein detection system, which meant that the detection objects could be both DNA and RNA; and there was no need for the reverse transcription process.

### Example 5: Nucleic Acid Amplification could Effectively Improve the Sensitivity of a Cas12-Chimeric Probe System

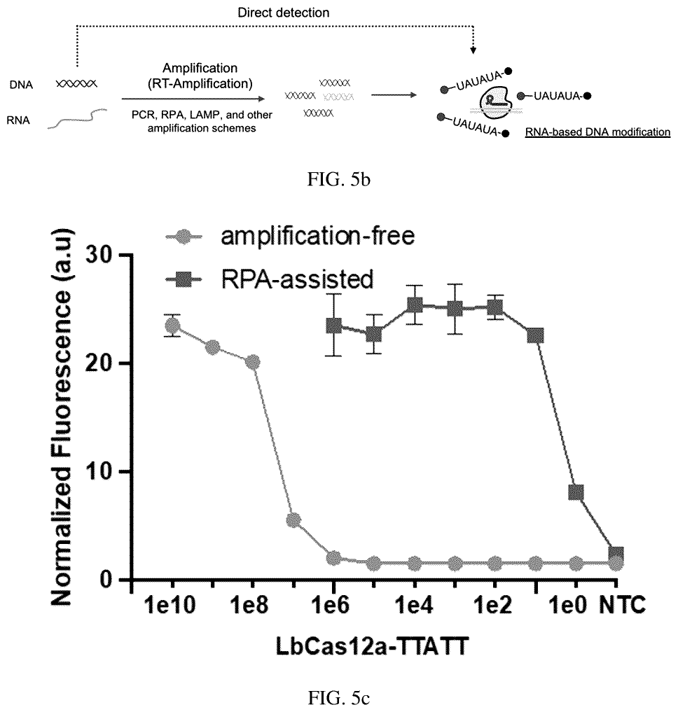

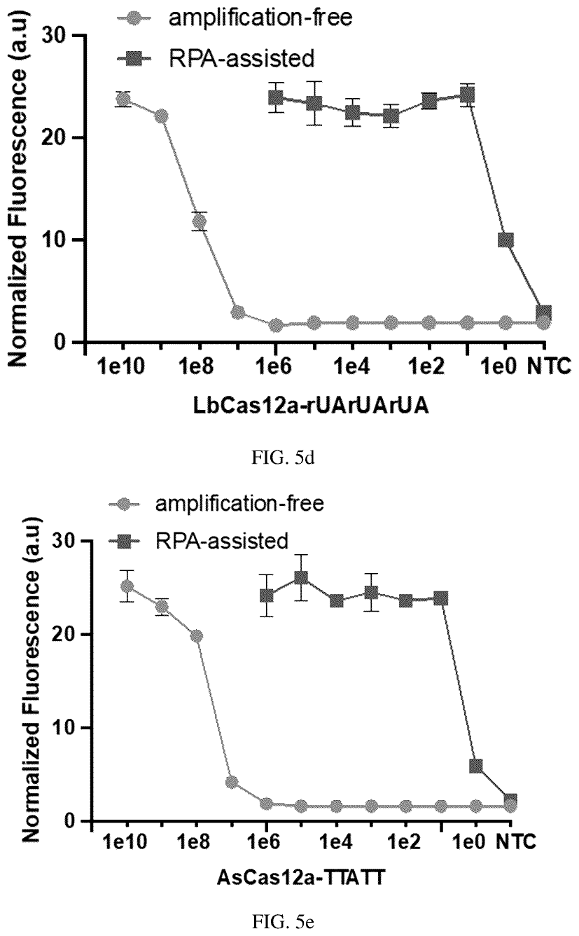

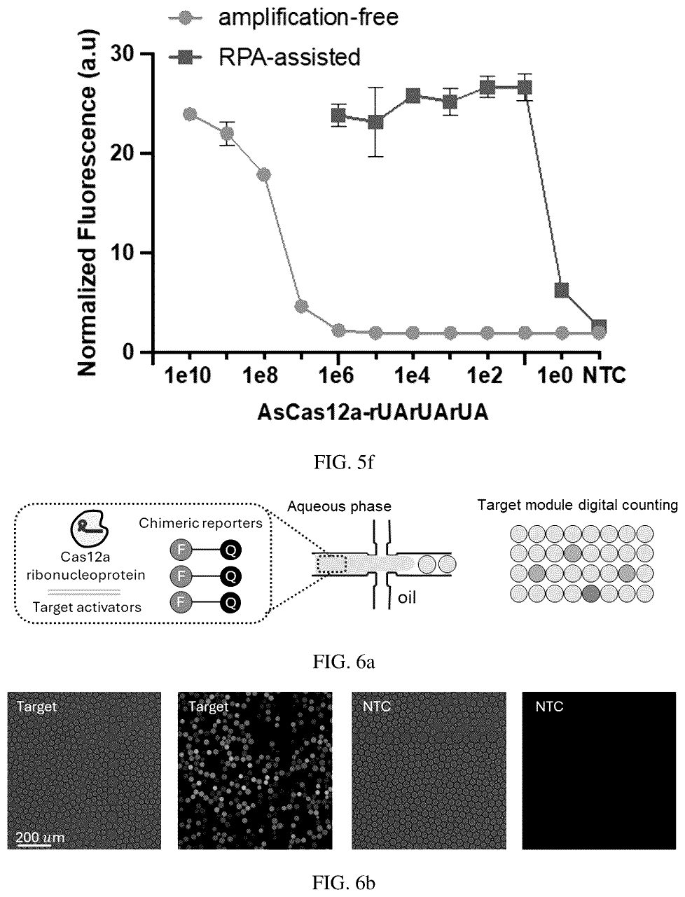

Examples 2-4 demonstrated the role of chimeric sequences in the Cas12 detection system, and also provided new schemes for the trans-cleavage of XNA, and optimization schemes for selecting different chimeric sequences according to different detection purposes. On the other hand, in the above examples (1-4), DNA or RNA was directly detected without amplification (amplification-free). In this example, a pre-amplification step was introduced to optimize the detection system of Cas12a-chimeric sequences. The pre-amplification in this example was recombinase polymerase amplification (RPA). However, it can be understood that any other method for amplifying target nucleic acids is still applicable. FIGS. 5a and 5b showed the flow charts for detecting target nucleic acid fragments by the pre-amplification or amplification-free methods. The target nucleic acid fragments included RNA and DNA. The probe used in FIG. 5a was the conventional ssDNA, such as TTATT. FIG. 5b showed the chimeric probe with an RNA backbone and DNA modifications, such as rUArUArUA. It can be understood that when the detection object was RNA, a reverse transcription reaction was required first, which meant that cDNA was synthesized using RNA as a template. For the specific transcription process, one could refer to the RPA reaction of relevant amplification method suppliers such as TwistAmp. Then an isothermal amplification method or polymerase chain reaction (PCR) was used to amplify the target fragment. The specific experimental steps were as follows: the RPA isothermal amplification was completed using the TwistAmp Basic kit, where the amplification primers were the same as that shown in SEQ ID NO: 27 and SEQ ID NO: 28, the total volume of the amplification reaction system was 20 μL, including 19 μL of the reaction solution (1 μL each of 10 μM forward and reverse primers, 12 μL of primer-free rehydration buffer, 1 μL of amplification template and 4 μL of ddH₂O) and 1 μL of 280 mM MgOAc initiator (final concentration 14 mM), the amplification template was the HPV18 sequence (SEQ ID NO: 1), the reaction temperature was 37° C., the reaction time was 20 minutes, and for the detailed reaction system and process, one could refer to the TwistAmp product manual; and 2 μL of the above amplified product was added into 18 μL of the Cas12a detection reaction solution to form a detection system with a total volume of 20 μL, where the components of the detection reaction solution and their total concentrations were as follows: 1×NEB 2.1 buffer, 50 nM Cas12a (LaCas12a/AsCas12a), 1 U/μL RNase inhibitor, 50 nM crRNA (SEQ ID NO: 8), and 125 nM probe (ssDNA (TTATT, SEQ ID NO: 2) or rUArUArUA (SEQ ID NO: 7)), and the fluorescence value was measured using a Roche Lightcycler 480 II instrument, where the set temperature was 37° C., the measurement was performed once every 30 s, and the measurement time is 30 min.

As can be seen from FIGS. 5c-f, under the amplification-free condition, the limits of detection of both the LbCas12a-rUArUArUA system (FIG. 5d) and the AsCas12a-rUArUArUA system (FIG. 5f) were the same as those of the LaCas12a/AsCas12a-TTATT (FIGS. 5c and 5e), reaching 1×10⁶ cp/μL. This result was consistent with the result of Example 2 mentioned above, indicating that the result stability of using chimeric sequences to detect target nucleic acids was relatively high; and after the detection samples were pretreated with RPA for 20 minutes, the detection sensitivities of the above 4 systems were improved by 1,000,000 times, reaching the single-molecule level. In summary, RPA could improve the detection level of the chimeric probe provided in the present invention, and on the other hand, it also indicated that the chimeric probe was applicable to conventional detection procedures.

### Example 6: Application of a Cas12a-Chimeric Sequence Detection System

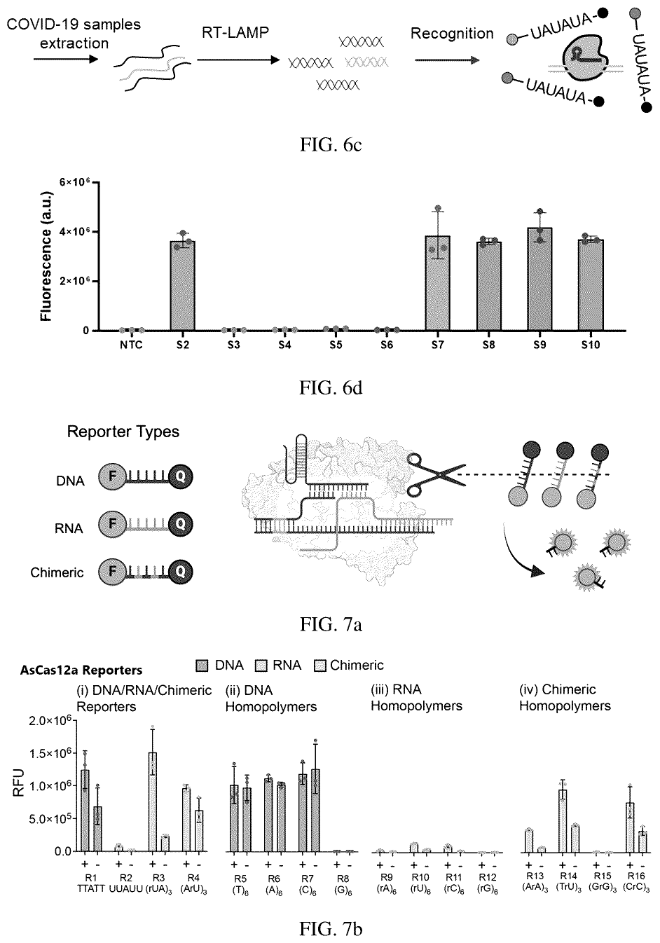

In order to further expand the application of the Cas12a-chimeric sequence detection system, in this example, the above system was combined with microfluidic technology to explore whether the Cas12a-chimeric sequence detection system was applicable to microarray reactions. The experimental principle is shown in FIG. 6a. The detection system flowed as the aqueous phase through the channels of the microfluidic chip, while the oil phase flowed into the channels from the other end. Subsequently, the aqueous phase and the oil phase were mixed. Since the aqueous phase and the oil phase were immiscible, individual droplets were formed. The experimental steps were as follows: on ice, a master mix with a total volume of 20 μL was prepared, which contained 1×NEB 2.1 buffer, 100 nM AsCas12a, 1 U/μL RNase inhibitor, 100 nM crRNA (SEQ ID NO: 8), 1 nM DNA for activating the Cas12 protein (SEQ ID NO: 1) (not added for the negative control), and 500 nM chimeric reporter (rUArUArUA, SEQ ID NO: 7), and then loaded onto the microfluidic chip for droplet dispensing, where the microfluidic chip was prepared according to the standard of SU-8 (MicroChem, Westborough, MA, USA), and the specific steps were the same as those described in the reference [23], and the oil phase (Evagreen Digital PCR oil (#1864034)) used in this experiment was purchased from Bio-rad Company; and 20 μL of the above main reaction solution was used as the aqueous phase to form droplets with a diameter of 35 μm within 2 minutes, the droplets were incubated at 37° C. for 1 h, and then the luminescence of the droplets was observed under a fluorescence microscope (Nikon).

The results were shown in FIG. 6b. Since the negative control (NTC) did not contain the DNA activator, no fluorescence could be detected. On the other hand, the droplets with green fluorescence observed in the experimental group indicated that the chimeric sequence could still be effectively cleaved by the Cas12a protein under microscale reaction conditions, indicating that the Cas12a-chimeric sequence detection system was applicable to microscale reaction conditions, which was beneficial for high-throughput detection.

In addition, in this example, the Cas12a-chimeric sequence system was applied to the detection of COVID-19. The specific steps were as follows (FIG. 6c): the saliva samples of the detection subjects were collected, and the RNA in the samples was extracted by using the RNA extraction kit QIAamp Viral RNA Mini Kit (Qiagen, 52906), and the samples were subjected to reverse transcription loop-mediated isothermal amplification (RT-LAMP) for 20 min [24], where the RT-LAMP experiment used the WarmStart LAMP 2× Master Mix (E1700S) from NEB Company, the total volume of each reaction system was 30 μL, the system contained 0.2 μM LAMP-F3 (SEQ ID NO: 29) and 0.2 μM LAMP-B3 (SEQ ID NO: 30), 1.6 μM LAMP-FIP (SEQ ID NO: 31) and 1.6 μM LAMP-BIP (SEQ ID NO: 32) primers, 0.4 μM LAMP-LF (SEQ ID NO: 33) and 0.4 μM LAMP-LB (SEQ ID NO: 34) primers, and 1.0 μL of the RNA of the detection subjects, the system was made up to 30 μL with ddH₂O; and the reaction was performed at 65° C. for 30 min. The amplification product was mixed with the master mixture containing 1×NEB buffer 2.1, 50 nM Cas12a (LbCas12a), 1 U/L RNase inhibitor, 50 nM crRNA (the specific sequence was the same as that described in SEQ ID NO: 35), and 500 nM chimeric probe (rUArUArUA, SEQ ID NO: 7). Then 15 μL of the mixture was aspirated and transferred into a 384-well plate. After incubation at 37° C. for 1 h, the final fluorescence value of the reaction system was measured. A negative control and 3 replicates were set in this experiment.

As shown in the results of FIG. 6d, all 5 pre-identified COVID-19 positive samples were accurately detected, which was consistent with the results of RT-PCR detection, which meant that the detection accuracy of the Cas12a-chimeric sequence system was as high as 100%. The QuantiNova Probe RT-PCR Kit (Qiagen, 208354) was used in the RT-qPCR experiment. This clinical verification further demonstrated the practical application of the chimeric sequence in the field of diagnosis.

### Example 7: A Nucleic Acid Detection System

Combined with the experimental results of Examples 1-6, this example provided a nucleic acid detection system. When the detection object was DNA, the reaction volume of the detection system was 10-100 μL, and the components and their final concentrations of the reaction system were as follows: 1×NEB 2.1 buffer, 50 nM Cas12, 50 nM crRNA, 10 U rat RNase inhibitor, 500 nM probe, and 1× the DNA sample to be detected (the concentration of the DNA sample was preferably greater than 1 pM), where when the Cas12 protein was LbCas12a protein, the probe used could be any one or more of TrUTrUTrU (SEQ ID NO: 17), rUArUArUA (SEQ ID NO: 7) and ArUArUArU (SEQ ID NO: 6); when the Cas12 protein was AsCas12a protein, the probe used could be any one or more of rUArUArUA (SEQ ID NO: 7) and ArUArUArU (SEQ ID NO: 6); and the crRNA sequence was designed according to the specific sequence of the detection object.

When the detection object was RNA, cDNA needed to be generated through reverse transcription reaction. The detection system for cDNA was the same as the DNA detection system described above (amplification was carried out in advance before detection). Alternatively, a DNA helper sequence could be introduced into the detection system to directly detect RNA (pre-amplification was not necessary). Specifically, the reaction volume of the detection system was 10-100 μL, and the components and their final concentrations of the reaction system were as follows: 1×NEB 2.1 buffer, 90 nM AsCas12a, 50 U RNase inhibitor, 20 nM DNA helper, 90 nM gRNA, 31.25 mM MgCl2, 500 nM probe (rUArUArUA), and 1× the RNA sample to be detected (the RNA sample concentration was preferably greater than 1 pM). The DNA helper included a PAM sequence and a partial spacer sequence. In some specific examples, the DNA helper was as shown in SEQ ID NO: 25 and SEQ ID NO: 26, and the crRNA sequence was designed according to the specific sequence of the detection object.

The above detection reaction system for detecting DNA or RNA could be used in combination with experiments such as isothermal amplification, PCR or RPA. Amplification could improve the detection sensitivity of the system.

Combined with Examples 1-6, this example provided a method for detecting nucleic acids using the system according to Example 7. The specific steps were as follows: DNA or RNA was extracted from the samples, and if the sample was RNA, the RNA needed to be reverse-transcribed into cDNA or the RNA was directly detected using the DNA helper and Cas12a protein (AsCas12a was preferred in this example). The detection reaction system was prepared according to the detection object (DNA or RNA). The specific system was the same as described above. 10 μL-20 μL of the above reaction solution was taken and quickly transferred to a 96- or 384-well plate, and the plate was placed in a real-time fluorescence quantitative PCR instrument. The reaction temperature was determined by the type of Cas12 protein (the reaction temperature for LbCas12a and AsCas12a was 37° C., and that for AapCas12b was 60° C.). The data was read once every 30 s-60 s. The reaction time was 30 min-2 h. After the reaction was completed, a real-time fluorescence curve was generated or the final fluorescence value was directly read. Whether the detection result was positive was determined according to the fluorescence value. Three replicates and a blank control were set for each experiment.

### Example 8: Use of Chimeric Sequences in an RAPID System

A RAPID (RNA/DNA Affinity Precision Innovative Diagnostics) system is a CRISPR/Cas12a nucleic acid diagnostic system uniquely developed by our research team. By introducing a nick into the target sequence, the activity of Cas12a to cleave the target sequence can be activated. Cas12a can directly target the sequence downstream of the nick site and perform cleavage. Meanwhile, it can reversely cleave the probe (natural or non-natural probe), so that the detection of the target sequence can be completed without relying on the PAM sequence. With the introduction of this gap or nick, the activity of Cas12a is activated, thereby enhancing the activity of its trans-cleavage sequence.

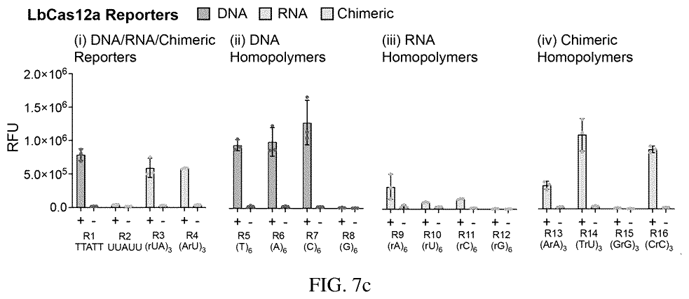

The “gap” or “nick” herein may be a nick on the target nucleic acid sequence itself, or a nick formed by binding of other sequences to the target sequence. As shown in the figure, when the protein binds to the target nucleic acid 101, 102 or 104, and when the guide sequence 100 binds to the target sequence, if one or more bases therein do not form a pair, a gap or nick is formed. The formation of such a gap can be achieved when the guide sequence directly combines with the target sequence (base non-pairing), or it can be achieved when the guide sequence combines with other sequences to form one or more nicks. As illustrated in FIG. 7a, the leader or guide sequence 100 forms a nick 105 together with sequences 101, 104. The target sequence herein can be a single-stranded or double-stranded nucleic acid sequence, such as DNA or RNA.

In order to improve the performance of RAPID and expand its application, in this example, the effect of using non-ssDNA probes for RAPID detection of different Cas12 proteins was investigated. The schematic diagram of the process is shown in FIG. 7a. Taking AsCas12a and LbCas12a as the research objects respectively. 3 different types of probes were selected, i.e. ssDNA probes, ssRNA probes and chimeric sequence probes (one of non-natural sequences), to investigate whether different probes could all be used for the trans-cleavage of the RAPID system to achieve detection.

Specifically, the target sequence with the gap site was the double-stranded binding product of DNA helper sense and DNA helper anti-sense (SEQ ID NO: 36, SEQ ID NO: 37), and the probes were labeled with the FAM fluorescent group and the 3IABKFQ quenching group, where the ssDNA probes included: R1 (TTATT) (SEQ ID NO: 2), R5 ((T)₆) (SEQ ID NO: 13), R6 ((A)₆) (SEQ ID NO: 10), R7 ((C)₆) (SEQ ID NO: 12), R8 ((G)₆) (SEQ ID NO: 11); the ssRNA probes included: R2 (UUAUU) (SEQ ID NO: 38), R9 ((rA)₆) (SEQ ID NO: 4), R10 ((rU)₆) (SEQ ID NO: 3), R11 ((rC)₆) (SEQ ID NO: 15), R12 ((rG)₆) (SEQ ID NO: 14); and the chimeric sequence probes included: R3 ((rUA)₃) (SEQ ID NO: 7), R4 ((ArU)₃) (SEQ ID NO: 6), R13 ((ArA)₃) (SEQ ID NO: 18), R14 ((TrU)₃) (SEQ ID NO: 17), R15 ((GrG)₃) (SEQ ID NO: 20), R16 ((CrC)₃) (SEQ ID NO: 19). The specific experimental operations were as follows: a 50 μL reaction system was prepared: 1×NEB 2.1 buffer (New England Biolab), 90 nM Cas12 (the types of Cas12 protein in this example were AsCas12a or LbCas12a), 90 nM gRNA of LbCas12a or AsCas12a (the specific sequences were as shown in SEQ ID NO: 39 and SEQ ID NO: 40), 50 U rat RNA enzyme inhibitor (RNase inhibitor Murine), 31.25 mM MgCl2 and 500 nM probe, then 50 nM of the target sequence with the gap site (SEQ ID NO: 36, SEQ ID NO: 37) was added, where the activated single-stranded sequence was (SEQ ID NO: 41), and in the negative control group, 2 μL of ddH₂O was substituted for the added target sequence, and they were mixed evenly; and 15 μL of the mixture was aspirated and transferred into a 384-well plate, and after incubation at 37° C. for 1 h, the final fluorescence value of the reaction system was measured, with 3 replicates set for each reaction system. The results were expressed as the mean±standard deviation (n=3) in relative fluorescence units (RFU). The bar graph represented the arithmetic mean+SD. The detection results are shown in FIG. 7b and FIG. 7c, where the performance detection results of RAPID when using AsCas12a and different probes are shown in FIG. 7b, and the performance detection results of RAPID when using LbCas12a and different probes are shown in FIG. 7c.

As could be seen from FIG. 7b and FIG. 7c, when the probe was the ssDNA sequence (R1: TTATT), the trans-cleavage performance of RAPID using AsCas12a and LbCas12a was consistent with that of the traditional Cas12a system. Compared with LbCas12a, AsCas12a showed a non-specific high signal in the absence of the target sequence (negative control group) ((i) in FIG. 7b and (i) in FIG. 7c). When the ssRNA sequence was used for trans-cleavage (R2: rUrUrArUrU), the RNase activity of AsCas12a was stronger than that of LbCas12a, but the relative signals of both were relatively weak. However, when DNA bases were introduced on the basis of ssRNA to construct chimeric sequences (R3: (rUA); and R4: (ArU) 3), surprisingly, the trans-cleavage signals of these chimeric sequences were significantly enhanced compared with the RNA-based sequences of AsCas12a and LbCas12a ((i) in FIG. 7b and (i) in FIG. 7c), and the background signal of AsCas12a could also be significantly reduced, especially when the probe was the chimeric sequence R3.

When ssDNA homopolymers (R5: (T)₆, R6: (A)₆, R7: (C)₆ and R8: (G)₆) were used as probes, the DNase activity was investigated in the presence and absence of the target sequence. It was found that compared with the high signal and low background of LbCas12a, the ssDNA homopolymers produced very high background signals for R5, R6 and R7 of AsCas12a ((ii) in FIG. 7b). Meanwhile, no trans-cleavage signals were observed when the probe was poly G (R8) in both AsCas12a and LbCas12a, which was similar to the results of Example 2.

When ssRNA homopolymers (R9: (rA)₆, R10: (rU)₆, R11: (rC)₆ and R12: (rG)₆) were used as probes, RAPID was tested to assess its RNase activity. The RNase activities of R10 and R11 in AsCas12a and R9. R10 and R11 in LbCas12a were relatively weak, and the activities of R9 and R12 with AsCas12a as well as R12 with LbCas12a were extremely low ((iii) in FIG. 7b and (iii) in FIG. 7c).

When RAPID was applied to the trans-cleavage of chimeric homopolymers (R13: (ArA)₃, R14: (TrU)₃, R15: (GrG)₃ and R16: (CrC)₃), R13, R14 and R16 had good trans-cleavage effects with AsCas12a and LbCas12a, while R15 was hardly cleaved ((iv) in FIG. 7b and (iv) in FIG. 7b).

The above results indicated that both chimeric sequence probes and RNA probes could be used for the detection of RAPID. The performance of chimeric sequence probes was comparable to that of some ssDNA sequences. For example, when using the chimeric sequence probe R3, the performance was enhanced and the non-specific background signal of AsCas12a was significantly reduced, indicating that AsCas12a had a wider tolerance for chimeric sequence probes than LbCas12a. In addition, the background signal from the ssDNA probe R1 (the traditional reporter substrate for Cas12a) was significantly higher than that of the chimeric sequence R3, indicating that the chimeric sequence probes could not only achieve the cleavage efficiency of the ssDNA probes, but also minimize the background interference, and the comprehensive effect was obviously better than that of the traditional ssDNA probes.

When LbCas12a was used, the ssDNA sequences also showed superior trans-cleavage activity compared with RNA probes, highlighting the inherent preference of LbCas12a for ssDNA probes in the RAPID system. The ability of LbCas12a to cleave the chimeric sequence probes R3 and R4 was slightly reduced compared with that of ssDNA R1, but its ability to cleave the chimeric sequence probes R14 and R16 was excellent, which also indicated that the RAPID system had flexible trans-cleavage substrate specificity at the same time.

In summary, when the traditional ssDNA probes were used, AsCas12a was generally affected by high background signals, and the chimeric sequence probes (such as R3) overcame this limitation. For LbCas12a, the chimeric sequence probes provided an effective scheme to improve the signal-to-noise ratio. Based on the nucleic acid detection of LbCas12a and AsCas12a in the RAPID system, combined with chimeric sequence probes (such as R3: (rUA) 3, etc.), the detection sensitivity of the target sequence could be further improved.

All patents and publications mentioned in the specification of the present invention indicate that they are published techniques in the art and can be used by the present invention. All patents and publications cited herein are also listed in the references as if each publication is specifically and individually cited. The present invention described herein may be practiced in the absence of any element or elements, limitation or limitations, and here such a limitation is not specifically stated. For example, in each instance here the terms “comprising/including”, “consisting essentially of” and “consisting of” may be replaced by one of the remaining 2 terms. The so-called “a/an” here only means “one”, and it does not exclude that it only includes one, or alternatively it can also mean including 2 or more. The terms and expressions adopted here are for description rather than limiting, and there is no intention to indicate that these terms and explanations described in this specification exclude any equivalent features, but it can be known that any suitable changes or modifications can be made within the scope of the present invention and the claims. It can be understood that the examples described in the present invention are some preferred examples and features. Some modifications and changes can be made by any person of ordinary skills in the art based on the essence of the description of the present invention, and these modifications and changes are also considered to belong to the scope of the present invention and the scope limited by the independent claims and the dependent claims.

Sequences used in this inventions as below table.

TABLE 3
sequences used in this inventon as an embodiement or example.

| | | Serial number of |
|---|---|---|
| Name | Sequence (5′-3′) | primer |
| HPV 18 | TTGTTACCTCTGACTCCCAGTTGTTTAATAAA | SEQ ID NO: 1 |
| | CCATATTGGTTACATAAGGCACAGGGTCATA | |
| | ACAATGGTGTTTGCTGGCATAATCAATTATTT | |
| | GTTACTGTGGTAGATACCACTCCCAGTACCAA | |
| | TTTAACAATATGTGCTTCTACACAGTCTCCTG | |
| | TACCTGGGCAATATGATGCTACCAAATTTAAG | |
| | CAGTATAGCAGACATGTTGAGGAATATGATTT | |
| | GCAGTTTATTTTTCAGTTGTGTACTATTACTTT | |
| | AACTGCAGATGTTATGTCCTATATTCATAGTA | |
| | TGAATAGCAGTA | |
| TTATT | /56-FAM/TTATT/3IABKFQ/ | SEQ ID NO: 2 |
| rUrUrUrUrUrU (poly rU) | /56-FAM/rUrUrUrUrUrU/3IABkFQ/ | SEQ ID NO: 3 |
| rArArArArArA (poly rA) | /56-FAM/rArArArArArA/3IABkFQ/ | SEQ ID NO: 4 |
| T*A*rArU*G*C | /56-FAM/T*A*rArU*G*C/3IABKFQ/ | SEQ ID NO: 5 |
| ArUArUArU (poly rAU) | /56-FAM/ArUArUArU/3IABKFQ/ | SEQ ID NO: 6 |
| rUArUArUA (poly rUA) | /56-FAM/rUArUArUA/3IABKFQ/ | SEQ ID NO: 7 |
| HPV 18-Lb12 and AsCas12- | ACAAUAUGUGCUUCUACACA | SEQ ID NO: 8 |
| spacer sequence | | |
| TTATTT | /56-FAM/TTATTT/3IABKFQ/ | SEQ ID NO: 9 |
| AAAAAA (poly A) | /56-FAM/AAAAAA/3IABKFQ/ | SEQ ID NO: 10 |
| GGGGGG (poly G) | /56-FAM/GGGGGG/3IABKFQ/ | SEQ ID NO: 11 |
| CCCCCC (poly C) | /56-FAM/CCCCCC/3IABKFQ/ | SEQ ID NO: 12 |
| TTTTTT (poly T) | /56-FAM/TTTTTT/3IABKFQ/ | SEQ ID NO: 13 |
| rGrGrGrGrGrG (poly rG) | /56-FAM/rGrGrGrGrGrG/3IABkFQ/ | SEQ ID NO: 14 |
| rCrCrCrCrCrC (poly rC) | /56-FAM/rCrCrCrCrCrC/3IABKFQ/ | SEQ ID NO: 15 |
| rUrUrArUrUrU | /56-FAM/rUrUrArUrUrU/3IABkFQ/ | SEQ ID NO: 16 |
| TrUTrUTrU (poly TrU) | /56-FAM/TrUTrUTrU/3IABkFQ/ | SEQ ID NO: 17 |
| ArAArAArA (poly ArA) | /56-FAM/ArAArAArA/3IABKFQ/ | SEQ ID NO: 18 |
| CrCCrCCrC (poly CrC) | /56-FAM/CrCCrCCrC/3IABKFQ/ | SEQ ID NO: 19 |
| GrGGrGGrG (poly GrG) | /56-FAM/GrGGrGGrG/3IABKFQ/ | SEQ ID NO: 20 |
| COVID-19 N sequence | UGCAACUGAGGGAGCCUUGAAUACACCAAAA | SEQ ID NO: 21 |
| | GAUCACAUUGGCACCCGCAAUCCUGCUAAC | |
| | AAUGCUGCAAUCGUGCUACAACUUCCUCAA | |
| | GGAACAACAUUGCCAAAAGGCUUCUACGCA | |
| | GAAGGGAGCAGAGGCGGCAGUCAAGCCUCU | |
| | UCUCGUUCCUCAUCACGUAGUCGCAACAGUU | |
| | CAAGAAAUUCAACUCCAGGCAGCAGUAGGG | |
| | GAACUUCUCCUGCUAGAAUGGCUGGCAAUG | |
| | GCGGUGAUGCUGCUCUUGCUUUGCUGCUGC | |
| | UUGACAGAUUGAACCAGCUUGAGAGCAAAA | |
| | UGUCUGGUAAAGGCCAACAACAACAAGGCC | |
| | AAACUGUCACUAAGAAAUCUGCUGCUGAGG | |
| | CUUCUAAGAAGCCUCGGCAAAAACGUACUG | |
| | CCACUAAAGCAUACAAUGUAACACAAGCUU | |
| | UCGGCAGACGUGGUCCAGAACAAACCCAAG | |
| | GAAAUUUUGGGGACCAGGAACUAAUCAGAC | |
| | AAGGAACUGAUUACAAACAUUGGCCGCAAA | |
| | UUGCACAAUUUGCCCCCAGCGCUUCAGCGU | |
| | UCUUCGGAAUGUCGCGCAUUGGCAUGGAAG | |
| | UCACACCUUCGGGAACGUGGUUGACC | |
| synthetic COVID-19 | CGAAGAACGCUGAAGCGCUG | SEQ ID NO: 22 |
| -N gene LbCas12a, | | |
| AsCas12a, AapCas12b | | |
| spacer sequence | | |
| RNA | UAGCUUAUCAGACUGAUGUUGA | SEQ ID NO: 23 |
| AsCas12a spacer | UGAAUCAACAUCAGUCUGAU | SEQ ID NO: 24 |
| DNA helper-sense | GAAGTTCATGTTTCTGAATCAACATCAGTCTG | SEQ ID NO: 25 |
| | ATAAGCTATTCAAG | |
| DNA helper-anti-sense | TTCAGAAACATGAACTTC | SEQ ID NO: 26 |
| HPV18-RPA-F | AATTATTTGTTACTGTGGTAGATACCACTCCC | SEQ ID NO: 27 |
| | AG | |
| HPV18-RPA-R | CACAACTGAAAAATAAACTGCAAATCATATT | SEQ ID NO: 28 |
| | CCTC | |
| LAMP-F3 | AGATCACATTGGCACCCG | SEQ ID NO: 29 |
| LAMP-B3 | CCATTGCCAGCCATTCTAGC | SEQ ID NO: 30 |
| LAMP-FIP | TGCTCCCTTCTGCGTAGAAGCCAATGCTGCAA | SEQ ID NO: 31 |
| | TCGTGCTAC | |
| LAMP-BIP | GGCGGCAGTCAAGCCTCTTCCCTACTGCTGCC | SEQ ID NO: 32 |
| | TGGAGTT | |
| LAMP-LF | GCAATGTTGTTCCTTGAGGAAGTT | SEQ ID NO: 33 |
| LAMP-LB | GTTCCTCATCACGTAGTCGCAACA | SEQ ID NO: 34 |
| COVID-19 LbCas12 spacer | UGAACCUCAUCACGUAGUCG | SEQ ID NO: 35 |
| RAPID-DNA helper-sense | GAAGTTCATGTTTCTGAAGTAGATATGGCAGC | SEQ ID NO: 36 |
| | ACTAATCTAATATG | |
| RAPID-DNA helper antisense | TTCAGAAACATGAACTTC | SEQ ID NO: 37 |
| UUAUU (R2) | /56-FAM/UUAUU/3IABKFQ/ | SEQ ID NO: 38 |
| AsCas12a crRNA spacer | UGAAGUAGAUAUGGCAGCAC | SEQ ID NO: 39 |
| LbCas12a crRNA spacer | UGAAGUAGAUAUGGCAGCAC | SEQ ID NO: 40 |
| ssDNA activator | GCGCTAATACGACTCACTATAGGGGCIGTCAT | SEQ ID NO: 41 |
| | TGATGCATATTAGATTAGTGCTGCCATATCTA | |
| | CAGGTCGACTTTCAAGAATTCATAT | |

### REFERENCES

[1] D. S. Chertow, Next-generation diagnostics with CRISPR. Science. 360, 381-382 (2018).
[2] M. M. Kaminski, O. O. Abudayyeh, J. S. Gootenberg, F. Zhang, J. J. Collins, CRISPR-based diagnostics. Nature Biomedical Engineering. 5, 643-656 (2021).
[3] S. Li et al., CRISPR-Cas12a-assisted nucleic acid detection. Cell Discovery. 4, 1-4 (2018).
[4] J. S. Chen et al., CRISPR-Cas12a target binding unleashes indiscriminate single-stranded DNase activity. Science. 360, 436-439 (2018).
[5] J. S. Gootenberg et al., Nucleic acid detection with CRISPR-Cas13a/C2c2. Science. 356, 438-442 (2017).
[6] F. X. Liu, J. Q. Cui, Z. Wu, S. Yao, Recent progress in isothermal nucleic acid detection with CRISPR. Lab on a Chip. (2023).
[7] Z. Li, H. Zhang, R. Xiao, R. Han, L. Chang, Cryo-EM structure of the RNA-guided ribonuclease Cas12g. Nature Chemical Biology. 17, 387-393 (2021).
[8] J. P. Bravo et al., RNA targeting unleashes indiscriminate nuclease activity of CRISPR-Cas12a2. Nature. 613, 582-587 (2023).
[9] S. R. Rananaware et al., Programmable RNA detection with CRISPR-Cas12a. Nature Communications. 14, 5409 (2023).
[10] R. T. Fuchs, J. Curcuru, M. Mabuchi, P. Yourik, G. B. Robb, Cas12a trans-cleavage can be modulated in vitro and is active on ssDNA, dsDNA, and RNA. BioRxiv., 600890 (2019).
[11] K. Morihiro, Y. Kasahara, S. Obika, Biological applications of xeno nucleic acids. Molecular BioSystems. 13, 235-245 (2017).
[12] Deng, F., Sang, R., Li, Y., Yang, D., Deng, W., & Goldys, E. (2024). Towards Understanding Trans-Cleavage of Natural and Synthetic Nucleic Acids by Cas12a for Sensitive CRISPR Biosensing.
[13] Li, B., Zeng, C., Li, W., Zhang, X., Luo, X., Zhao, W., . . . & Dong, Y. (2018). Synthetic oligonucleotides inhibit CRISPR-Cpf1-mediated genome editing. Cell reports, 25 (12), 3262-3272.
[14] Ke, Y., Ghalandari, B., Huang, S., Li, S., Huang, C., Zhi, X., . . . & Ding, X. (2022). 2′-O-Methyl modified guide RNA promotes the single nucleotide polymorphism (SNP) discrimination ability of CRISPR-Cas12a systems. Chemical Science, 13 (7), 2050-2061.
[15] J. Joung et al., Detection of SARS-COV-2 with SHERLOCK one-pot testing. N. Engl. J. Med. 383, 1492-1494 (2020).
[16] J. Q. Cui et al., Droplet digital recombinase polymerase amplification (ddRPA) reaction unlocking via picoinjection. Biosensors and Bioelectronics., 114019 (2022).
[17] M. Karlikow et al., CRISPR-induced DNA reorganization for multiplexed nucleic acid detection. Nature Communications. 14, 1505 (2023).
[18] J. S. Gootenberg et al., Multiplexed and portable nucleic acid detection platform with Cas13, Cas12a, and Csm6. Science. 360, 439-444 (2018).
[19] K. S. Makarova et al., Evolutionary classification of CRISPR-Cas systems: a burst of class 2 and derived variants. Nature Reviews Microbiology. 18, 67-83 (2020).
[20] M. J. Kellner, J. G. Koob, J. S. Gootenberg, O. O. Abudayyeh, F. Zhang, SHERLOCK: nucleic acid detection with CRISPR nucleases. Nature Protocols. 14, 2986-3012 (2019).
[21] H. de Puig et al., Minimally instrumented SHERLOCK (miSHERLOCK) for CRISPR-based point-of-care diagnosis of SARS-COV-2 and emerging variants. Science Advances. 7, eabh2944 (2021).
[22] Y. Zhao, F. Chen, Q. Li, L. Wang, C. Fan, Isothermal amplification of nucleic acids. Chem. Rev. 115, 12491-12545 (2015).
[23] F. X. Liu et al., Isothermal Background-Free Nucleic Acid Quantification by a One-Pot Cas13a Assay Using Droplet Microfluidics. Anal. Chem. (2022).
[24] J. Song et al., Smartphone-based SARS-COV-2 and variants detection system using colorimetric DNAzyme reaction triggered by loop-mediated isothermal amplification (LAMP) with clustered regularly interspaced short palindromic repeats (CRISPR). ACS Nano. 16, 11300-11314 (2022).

1. A probe comprising a non-natural sequence, wherein the non-natural sequence comprises any one or more of the following:
(1) a sequence containing both a deoxynucleotide and a ribonucleotide;
(2) a sequence containing a deoxynucleotide and/or ribonucleotide, wherein the deoxynucleotide and/or ribonucleotide bears an artificially created modification that does not exist under natural conditions; and
(3) a sequence containing a deoxynucleotide and/or ribonucleotide, wherein a backbone composed of the deoxynucleotide and/or ribonucleotide bears an artificially created modification that does not exist under natural conditions.

2. The probe according to claim 1, wherein the probe is composed of nucleotides constituting RNA and nucleotides constituting DNA, the nucleotides constituting the RNA are any one or more of a uracil ribonucleotide, an adenine ribonucleotide, a cytosine ribonucleotide and a guanine ribonucleotide, and the nucleotides constituting the DNA are any one or more of a thymine deoxynucleotide, an adenine deoxynucleotide, a cytosine deoxynucleotide and a guanine deoxynucleotide.

3. The probe according to claim 1, wherein the probe is arranged in the order of ribonucleotide-deoxynucleotide or deoxynucleotide-ribonucleotide, the ribonucleotide is any one or more of a uracil ribonucleotide, an adenine ribonucleotide, a cytosine ribonucleotide and a guanine ribonucleotide, and the deoxynucleotide is any one or more of a thymine deoxynucleotide, an adenine deoxynucleotide, a cytosine deoxynucleotide and a guanine deoxynucleotide.

4. The probe according to claim 1, wherein the probe comprises any one or more of rUArUArUA, ArUArUArU, rUrUrArUrUrU, TrUTrUTrU, ArAArAArA, CrCCrCCrC and GrGGrGGrG, wherein the rU is a uracil ribonucleotide, the A is an adenine deoxynucleotide, the T is a thymine deoxynucleotide, the rA is an adenine ribonucleotide, the C is a cytosine deoxynucleotide, the rC is a cytosine ribonucleotide, the G is a guanine deoxynucleotide, and the rG is a guanine ribonucleotide.

5. The probe according to claim 1, wherein the probe comprises any one or more of rUArUArUA, ArUArUArU and TrUTrUTrU.

6. The probe according to claim 1, wherein the probe is capable of being trans-cleaved by the Cas12 protein.

7. A kit for detecting a target nucleic acid in a sample, comprising: a Cas12 protein and a non-natural sequence capable of being trans-cleaved by the Cas12 protein.

8. The kit according to claim 7, wherein the Cas12 protein comprises Cas12a and Cas12b subfamilies.

9. The kit according to claim 7, wherein the Cas12 protein is any one or more of LbCas12a, AsCas12a and AapCas12b.

10. The kit according to claim 7, wherein the non-natural sequence comprises any one or more of the following:
(1) a sequence containing both a deoxynucleotide and a ribonucleotide;
(2) a sequence containing a deoxynucleotide and/or ribonucleotide, wherein the deoxynucleotide and/or ribonucleotide bears an artificially created modification that does not exist under natural conditions; and
(3) a sequence containing a deoxynucleotide and/or ribonucleotide, wherein a backbone composed of the deoxynucleotide and/or ribonucleotide bears an artificially created modification that does not exist under natural conditions.

11. The kit according to claim 7, wherein the non-natural sequence contains both the deoxynucleotide and the ribonucleotide.

12. The kit according to claim 11, wherein the deoxynucleotides and ribonucleotides in the non-natural sequence are arranged alternately at intervals.

13. The kit according to claim 7, wherein the non-natural sequence comprises a sequence composed of nucleotides constituting RNA and nucleotides constituting DNA, the nucleotides constituting the RNA are any one or more of a uracil ribonucleotide, an adenine ribonucleotide, a cytosine ribonucleotide and a guanine ribonucleotide, and the nucleotides constituting the DNA are any one or more of a thymine deoxynucleotide, an adenine deoxynucleotide, a cytosine deoxynucleotide and a guanine deoxynucleotide.

14. The kit according to claim 7, wherein the non-natural sequence is arranged in the order of ribonucleotide-deoxynucleotide or deoxynucleotide-ribonucleotide, the ribonucleotide is any one or more of a uracil ribonucleotide, an adenine ribonucleotide, a cytosine ribonucleotide and a guanine ribonucleotide, and the deoxynucleotide is any one or more of a thymine deoxynucleotide, an adenine deoxynucleotide, a cytosine deoxynucleotide and a guanine deoxynucleotide.

15. The kit according to claim 7, wherein the non-natural nucleic acid sequence comprises a (rUA) n nucleic acid sequence, wherein n is any natural integer.

16. The kit according to claim 15, wherein n=1-100.

17. The kit according to claim 7, wherein the non-natural sequence comprises any one or more of rUArUArUA, ArUArUArU, rUrUrArUrUrU, TrUTrUTrU, ArAArAArA, CrCCrCCrC and GrGGrGGrG, wherein the rU is a uracil ribonucleotide, the A is an adenine deoxynucleotide, the T is a thymine deoxynucleotide, the rA is an adenine ribonucleotide, the C is a cytosine deoxynucleotide, the rC is a cytosine ribonucleotide, the G is a guanine deoxynucleotide, and the rG is a guanine ribonucleotide.

18. The kit according to claim 17, wherein the non-natural sequence comprises any one or more of rUArUArUA, ArUArUArU and TrUTrUTrU.

19. The kit according to claim 7, further comprising a necessary agent for amplifying the target nucleic acid.

20. The kit according to claim 19, wherein the necessary agent comprises an enzyme, a primer, and an inorganic salt agent.

21. The kit according to claim 7, wherein the target nucleic acid comprises natural DNA or natural RNA.

22. The kit according to claim 21, wherein the DNA or RNA in the target nucleic acid comprises a double strand or a single strand.

23. The kit according to claim 21, wherein in the target nucleic acid, the DNA is double-stranded and the RNA is single-stranded.

24. The kit according to claim 7, wherein the target nucleic acid comprises a target nucleic acid with a nick.

25. The kit according to claim 7, wherein the non-natural sequence comprises a labeling substance, and the labeling substance is a fluorescent labeling substance.

26. The kit according to claim 7, wherein the non-natural sequence comprises a chimeric sequence.

27. The kit according to claim 7, further comprising an agent for reversely transcribing RNA into cDNA, or a DNA helper.

28. The kit according to claim 26, wherein the chimeric sequence comprises: a single chimera (poly ArA), a double chimera (poly rUArUA), and a multiple chimera (UrACrGTrA).

29. The kit according to claim 7, wherein the non-natural sequence comprises a nucleic acid sequence incapable of being produced or incapable of being stably inherited in a long evolutionary process in nature.

30. The kit according to claim 1, wherein the target nucleic acid is present in a sample.

31. The kit according to claim 7, wherein when the Cas12 protein is LbCas12a, the probe sequence comprises any one or more of rUArUArUA, ArUArUArU and TrUTrUTrU; and when the Cas protein is AsCas12a, the probe sequence comprises any one or more of rUArUArUA and ArUArUArU.

32. The kit according to claim 27, wherein the DNA helper is a partial sequence or a simple PAM sequence bearing a PAM sequence and capable of binding to target RNA.

33. The kit according to claim 24, wherein the nick includes by the target nucleic acid sequence itself, or the nick formed by binding of other sequences to the target sequence.

34. A method for detecting a target nucleic acid, comprising:
allowing a Cas12 protein to bind with the target nucleic acid; and
allowing the Cas12 protein to trans-cleave a non-natural sequence, so as to indicate presence or quantity of the target nucleic acid by the number of cleaved non-natural sequences.

35. The method according to claim 34, wherein the type of the target nucleic acid is natural DNA or natural RNA.

36. The method according to claim 34, wherein the Cas12 protein comprises Cas12a and Cas12b subfamilies.

37. The method according to claim 36, wherein the Cas12 protein is any one or more of LbCas12a, AsCas12a and AapCas12b.

38. The method according to claim 34, wherein the non-natural sequence comprises any one or more of the following:
(1) a sequence containing both a deoxynucleotide and a ribonucleotide;
(2) a sequence containing a deoxynucleotide and/or ribonucleotide, wherein the deoxynucleotide and/or ribonucleotide bears an artificially created modification that does not exist under natural conditions; and
(3) a sequence containing a deoxynucleotide and/or ribonucleotide, wherein a backbone composed of the deoxynucleotide and/or ribonucleotide bears an artificially created modification that does not exist under natural conditions.

39. The method according to claim 34, wherein the non-natural sequence contains both the deoxynucleotide and the ribonucleotide.

40. The method according to claim 39, wherein the deoxynucleotides and ribonucleotides in the non-natural sequence are arranged alternately at intervals.

41. The method according to claim 34, wherein the non-natural sequence comprises a sequence composed of nucleotides constituting RNA and nucleotides constituting DNA, the nucleotides constituting the RNA are any one or more of a uracil ribonucleotide, an adenine ribonucleotide, a cytosine ribonucleotide and a guanine ribonucleotide, and the nucleotides constituting the DNA are any one or more of a thymine deoxynucleotide, an adenine deoxynucleotide, a cytosine deoxynucleotide and a guanine deoxynucleotide.

42. The method according to claim 34, wherein the non-natural sequence is arranged in the order of ribonucleotide-deoxynucleotide or deoxynucleotide-ribonucleotide, the ribonucleotide is any one or more of a uracil ribonucleotide, an adenine ribonucleotide, a cytosine ribonucleotide and a guanine ribonucleotide, and the deoxynucleotide is any one or more of a thymine deoxynucleotide, an adenine deoxynucleotide, a cytosine deoxynucleotide and a guanine deoxynucleotide.

43. The method according to claim 34, wherein the non-natural nucleic acid sequence comprises a (rUA)ₙ nucleic acid sequence, wherein n is any natural integer.

44. The method according to claim 43, wherein n=1-100.

45. The method according to claim 34, wherein the non-natural sequence comprises any one or more of rUArUArUA, ArUArUArU, rUrUrArUrUrU, TrUTrUTrU, ArAArAArA, CrCCrCCrC and GrGGrGGrG, wherein the rU is a uracil ribonucleotide, the A is an adenine deoxynucleotide, the T is a thymine deoxynucleotide, the rA is an adenine ribonucleotide, the C is a cytosine deoxynucleotide, the rC is a cytosine ribonucleotide, the G is a guanine deoxynucleotide, and the rG is a guanine ribonucleotide.

46. The method according to claim 45, wherein the non-natural sequence comprises any one or more of rUArUArUA, ArUArUArU and TrUTrUTrU.

47. The method according to claim 34, wherein the target nucleic acid is amplified before bound by the Cas12 protein.

48. The method according to claim 47, wherein a method for the amplification comprises PCR or isothermal amplification.

49. The method according to claim 34, wherein when the target sequence is natural RNA, a DNA helper is added into the reaction and direct targeted binding of the Cas12 protein to the natural RNA is allowed.

50. The method according to claim 49, wherein when the target sequence is natural RNA, the natural RNA is reverse-transcribed into DNA.

51. The method according to claim 34, wherein before the target nucleic acid is bound by the Cas12 protein, when the target nucleic acid is present in the sample, the tested sample is processed and the target nucleic acid is extracted.

52. The method according to claim 34, wherein the target sequence is provided with a nick.

53. The method according to claim 52, wherein the nick includes by the target nucleic acid sequence itself, or the nick formed by binding of other sequences to the target sequence.

54. The method according to claim 52, wherein the target nucleic acid comprises natural DNA or natural RNA.

55. The method according to claim 54, wherein the DNA or RNA in the target nucleic acid comprises a double strand or a single strand.

56. The method according to claim 52, wherein in the target nucleic acid, the DNA is double-stranded and the RNA is single-stranded.

57. The method according to claim 34, wherein the non-natural sequence comprises a labeling substance, the labeling substance is a fluorescent labeling substance, and the presence or quantity of the target nucleic acid is indicated by the intensity of fluorescence.

58. The method according to claim 34, wherein the non-natural sequence comprises a chimeric sequence.

59. The method according to claim 58, wherein the chimeric sequence comprises: a single chimera (poly ArA), a double chimera (poly rUArUA), and a multiple chimera (UrACrGTrA).

60. The method according to claim 34, wherein the non-natural sequence comprises a nucleic acid sequence incapable of being produced or incapable of being stably inherited in a long evolutionary process in nature.
